(19) United States
(12) Patent Application Publication
(10) Pub. No.: US 2024/0011015 A1
(43) Pub. Date: Jan. 11, 2024

(54) ANTIGEN DISPLAY SYSTEM AND METHODS FOR CHARACTERIZING ANTIBODY RESPONSES

(71) Applicant: Duke University, Durham, NC (US)

(72) Inventors: Thomas F. TEDDER, Durham, NC (US); Evgueni KOUNTIKOV, Durham, NC (US)

(21) Appl. No.: 17/992,727

(22) Filed: Nov. 22, 2022

Related U.S. Application Data
(63) Continuation of application No. 16/493,243, filed on Sep. 11, 2019, now abandoned.
(60) Provisional application No. 62/470,667, filed on Mar. 13, 2017.

(51) Int. Cl. C12N 15/10; C12N 7/00

(52) U.S. Cl. CPC C12N 15/1037; C12N 7/00; C12N 2795/14121; C12N 2795/14131

(57) ABSTRACT

Provided herein is an antigen display library for detecting antibodies produced by an individual; and methods of using the antigen display library to generate an antibody signature, the method comprising contacting a biological sample containing antibodies from an individual with the antigen display library, isolating phage clones displaying antigenic epitopes recognized by antibody in the sample, and identifying the antigenic epitopes that were recognized by antibody in the sample. Also provided are kits for generating an antibody signature comprising the antigen display library, a substrate for isolating phage clones bound by antibody, and may further comprise reagents useful for generating the antibody signature.

### CROSS-REFERENCE TO RELATED PATENT APPLICATIONS

The present application is a continuation of U.S. application Ser. No. 16/493,243 filed Sep. 11, 2019, which is a national stage filing under 35 U.S.C. 371 of International Application No. PCT/US2018/022213, filed Mar. 13, 2018, which claims the benefit of priority of U.S. Provisional Patent Application No. 62/470,667, filed on Mar. 13, 2017, both of which are incorporated herein by reference in their entirety.

### SEQUENCE LISTING

This application is being filed electronically via Patent Center and includes an electronically submitted Sequence Listing in .xml format. The .xml file contains a sequence listing entitled “155554.00683.xml” created on Sep. 27, 2023 and is 25,683 bytes in size. The Sequence Listing contained in this .xml file is part of the specification and is hereby incorporated by reference herein in its entirety.

### TECHNICAL FIELD

The invention relates to compositions and methods for antigen display and for characterization of antibodies produced as a result of an individual's humoral immune response, including antibodies which recognize conformational epitopes. The characterization of antibodies produced by a humoral immune response can be used to generate signatures useful to identify a disease process, or to identify one or more antibodies or antigens that have potential diagnostic, prognostic, therapeutic, or theranostic applications. Additionally, an antibody signature (such as a computer-generated image) may be used to identify or subtype a disease process, which characteristically, is identified by such antibody signature.

### INTRODUCTION

Antibodies play important roles in both protective immune responses (e.g., immunity) and in pathogenic immune responses (e.g., autoimmunity). Disease processes, such as a microbial infection, an autoimmune disease, or cancer, expose the immune system to a distinct repertoire of antigens. In response, the humoral immune system generates a repertoire of antibodies shaped by such antigen exposure. Characterization of these antibody responses can provide important information on protective immune responses, as well as autoimmune responses, including identifying antibodies, or signatures comprised of multiple antibody responses, that could be developed as biomarkers or used for prognostic, diagnostic, theranostic, or therapeutic applications. There are a number of challenges in a method of characterizing such antibody responses. For example, in humans, the diversity and number of antibodies is very large. Additionally, a system to display epitopes of a large repertoire of antigens is needed. There is also a need to display these epitopes in a way that represents how an antigen is presented to and recognized by the humoral immune system.

Current technology uses peptide microarrays (e.g., peptides immobilized on a non-biological substrate) comprising a length of typically between about 15 to 30 amino acids, or T7 phage containing sequences of around 108 nucleotides and encoding peptides of 36 amino acids. These may be suitable for identifying antibodies that recognize linear epitopes on protein antigens. Linear epitopes are formed by a contiguous sequence of amino acids from an antigen that interact with an antibody's paratope, also called an antigen-binding site. Typically, a linear epitope is a contiguous sequence of amino acids and ranges from 5 to 8 amino acids in length. However, it has been estimated that more than 90% of B-cell epitopes are comprised of non-contiguous amino acids that are geometrically clustered due to molecular folding of the protein antigen, and are known in the art as conformational epitopes. The average amino acid sequence, comprising all amino acids for antibody contact and binding, and required for proper folding of a conformational epitope in native antigens, typically ranges from about 40 amino acids to about 600 amino acids, with the majority (90%) comprised of between 100 amino acid residues and 200 amino acid residues. The development of additional ways to characterize the breadth and diversity of antibodies produced by a humoral immune response is needed, including the generation of antibody signatures useful to identify a disease process.

### SUMMARY OF THE INVENTION

The invention is based on the development of an antigen display system that comprises Ff phage (filamentous phage that infect gram negative bacteria bearing the F episome) for the expression and presentation of linear epitopes and conformational epitopes, and its use to characterize antibody responses to complex mixtures of antigens.

In one aspect, Ff phage were used to construct the antigen display system to fit larger DNA fragments for expressing and presenting linear epitopes and conformational epitopes, and used to characterize antibody responses to the antigens, in overcoming limitations of the T7 phage system.

In one aspect, an antigen display system comprising an M13-based phage library is provided. The phage library comprises a plurality of phage clones containing cDNAs reverse transcribed from mRNA isolated from one or more cell types, cells from one or more tissue types (disease-specific or healthy tissues), cells from one or more organs, or a pool of phage libraries (each derived from mRNA isolated from a cell type or tissue type which is different than that from which other phage libraries in the pool are derived; “or combinations thereof”) from a mammal. In one aspect, the antigen display library contains clones that are representative of a substantial repertoire of antigenic epitopes expressed by the individual. In another aspect, the diversity of antigenic epitopes or polypeptides in the antigen display library is estimated to be greater than 1×10⁶, and in another aspect greater than 3×10⁷. Prior to cloning the cDNA into the phage vector in constructing the phage library, the cDNA is selected for a size ranging of from about 150 nucleotides to about 900 nucleotides in length to facilitate detection of sequences that encode linear epitopes and conformational epitopes. The size-selected cDNA is selected for in-frame cDNA fragments by directional molecular cloning into a plasmid comprising a selectable marker to allow the positive selection of transformed cells so that only insert-encoded polypeptides that were in-frame with a selectable marker (e.g., plasmid β-lactamase gene) at the 3′ end of the cDNA insert would be expanded during plasmid library amplification. This intermediate cloning step allows for nine-fold enrichment in polypeptides that represent native mRNA-encoded amino acid species. The cDNA from this intermediate cloning step was cloned into M13 phage in constructing the phage library.

In some embodiments, the DNA inserts in the antigen display libraries described herein do not have to be derived from an mRNA (i.e., be a cDNA). For example, the DNA inserts may be derived from any source. Exemplary sources may include, without limitation, synthetic gene libraries. Accordingly, in another aspect, the present invention relates to an antigen display library including a Ff phage-based library comprised of a plurality of phage clones containing a plurality of DNA inserts inserted therein, wherein the DNA inserts: (a) each encode a polypeptide; (b) comprise an average length selected from between about 150 nucleotides and about 900 nucleotides; and (c) are selected for in-frame expression of the polypeptide.

In one aspect, the phage library is contacted with a sample of body fluid from an individual, containing or suspected of containing antibody. Recombinant phage expressing and displaying antigenic epitopes which are recognized by antibodies (e.g., antibodies have binding specificity for such displayed antigens) in the sample become bound to the antibody. The antibodies in the sample may be immobilized to a substrate to facilitate isolation of recombinant phage expressing and displaying antigens to which the antibodies are bound. The methods of the present invention allow for the interaction of antibody with antigen in solution, thereby preserving the secondary and tertiary domain structure of the protein comprising the antigen, as compared to assays that depend on the attachment or capture of the antigen on a solid surface.

To identify the antigenic epitopes, the method may further comprise isolating the recombinant phage expressing and displaying antigenic epitopes which are recognized by the antibodies, and sequencing the inserts from such recombinant phage to identify the antigens (via the nucleotide sequence of the gene or portion thereof encoding such antigen). The method obviates the use of secondary antibody or other means to detect the primary antibody in the process of identifying the antigens. The method may further comprise using bioinformatics to sort the gene and protein sequences identified in this method into categories or distributions based on certain parameters (e.g., one or more of abundance of expression or occurrence, diversity of expression, relatedness of antigens, identification of self-antigens, identification of foreign antigens, functional or metabolic groups, co-isolation using the same antibody sample, nucleotide or amino acid sequences, homology to nucleotide or protein sequences found within specific cells, genes, or the genomes of different species or organisms, or homology to nucleotide sequences found within specific diseased or malignant cells or tissues) in generating a profile or signature of antibody responses to such antigens. These profiles or signatures can be compared between individuals and may be developed as biomarkers or for prognostic, diagnostic or therapeutic applications. The method allows the simultaneous identification of approximately 20,000 or more antigens, and about 5,000,000 or more antigen fragments identified by antibodies in a single sample of human serum. Analysis identifies the gene product recognized by antibodies, and also quantifies the domains of the protein product containing one or more antigenic epitopes that are identified by antibodies, allowing for epitope mapping and in the case of autoimmune disease, the analysis of epitope spreading during the course of disease development and progression.

In another aspect, antibodies in the sample from the individual may comprise IgA, IgM, IgE, and IgG antibodies. In a further aspect, the substrate for immobilizing antibody may be selective for binding one subclass of immunoglobulin (e.g., IgG), or more than one subclass of immunoglobulin, which is then contacted with the recombinant phage. Alternatively, one or more immunoglobulin subclasses may be purified from the sample prior to contact with the recombinant phage library, and which is then used to contact the recombinant phage. In one aspect, IgG is used to contact the recombinant phage. In a further aspect of the invention, the method may be used to determine the identity or diversity of antigens recognized by a monoclonal antibody or resulting from a polyclonal antibody response after antigen, vaccine, or pathogen challenge.

In one aspect, the antigen display system and methods of use thereof, can be used to measure complex antibody responses to antigens comprising self-antigens, neoantigens, and cancer antigens. In another aspect, the antibody response measured may be to antigens comprising microbial antigens. Such measurement can also take place following immunotherapy (e.g., vaccination) for assessing a change in such antibody response (e.g., comparing the antibody response prior to immunotherapy with the antibody response following immunotherapy). Such measurement can be used to identify antigens that may be used to confer protective immunity. Such measurements can also be used to identify self-antigens that play an important role in a pathologic immune response (e.g., that induces or regulates a disease process comprising autoimmunity, allergy, inflammation, transplantation rejection). Further, such measurements may be arranged in a pattern of antigens recognized in generating an image represented by one or more parameters comprising frequency of detection, size of antigenic epitope, diversity of expression, relatedness in sequence to other antigens detected, relatedness as to expression in the same disease process, identification of self-antigens, nucleotide sequences or homology to nucleotide sequences found within specific cells, genes, or the genomes of different species or organisms, or homology to nucleotide sequences found within specific diseased or malignant cells.

Provided is a method of determining an antibody signature by analyzing a sample obtained from an individual with an immune-related disease, the method comprising contacting an antigen display library provided herein with the sample comprising antibodies; identifying antigens which are bound by the antibodies; and generating an antibody signature based on the antigens identified from binding by antibody in the sample obtained from the individual with an immune-related disease.

The method may further comprise amplifying the phage clones bound by antibody prior to identifying the antigenic epitopes recognized by antibody in the sample. The phage clones bound by antibody may be amplified, for example, by infecting a cell line capable of supporting the replication of the phage clones such as, without limitation, TG1 cells.

The method may further comprise comparing an antibody signature generated from analysis of a sample obtained from an individual with an immune-related disease with an antibody signature generated from a sample obtained from an individual not known to have an immune-related disease (e.g., healthy individual) in identifying antigens associated with such immune-related disease as compared to absence of such immune-related disease (occurring in a statistically significant higher frequency of detection by antibody generated from the immune-related disease, as compared to detection by antibody generated in the absence of such disease). Where an antigen is identified as specific for or associated with an immune-related disease, and genetic sequence analysis identifies the antigen as a self-antigen, the antibody signature may comprise an autoantibody signature. Comparisons may be made between two or more antibody signatures generated from samples obtained from the same individual, or may be made between two or more antibody signatures generated from samples obtained from individuals known or suspected to have the same disease process, or may be made between two or more antibody signatures generated from samples obtained from individuals known or suspected to have different disease processes as compared to each other. Antibody signatures may be separated by cohorts for comparison purposes. Antibody signatures can be used to assess disease (by changes in induction of antibody by antigens) at various stages of diagnosis, progression or prognosis, which can be used for comparison between samples from a single individual or between different individuals. For example, some autoantibodies are disease-specific, some associate with distinct disease subtypes and with differences in disease severity, and may be correlated with genetic, demographic, diagnostic, clinical, and prognostic aspects of autoimmune disease. In many cases, serum autoantibodies may even precede the onset of autoimmune disease by several years.

In another aspect, provided is a method for identifying protein:protein interactions and isolating interacting proteins from the complex mixture of protein domains expressed by the phage library. In one example, the expressed protein domains expressed within the phage display library may serve as a ligand for a cell surface or intracellular receptor.

In another aspect, provided is a kit for detecting antibodies, in a sample from an individual, which recognize and bind to antigenic epitopes expressed by the antigen display system provided herein, wherein the kit comprises phage comprising the antigen display system provided herein, a substrate to which the user may bind antibodies present in the sample, and packaging for holding the phage and for holding the substrate. The substrate may be provided as a premade affinity substrate, or may contain the substrate and affinity reagent as separate components for the user to combine. The kit may further comprise one or more reagents necessary for binding antibodies to the substrate to produce an affinity substrate, or for contacting the phage with the antibodies present in the sample, or for nucleic acid amplification of nucleic acid sequences encoding antigenic epitopes displayed by the phage and recognized by antibody in the sample.

### BRIEF DESCRIPTION OF THE FIGURES

FIG. 1A is a schematic diagram summarizing production of the phage display library for the expression and presentation of linear epitopes and conformational epitopes, and its use to characterize antibody responses to the antigens.

FIG. 1B is a schematic diagram showing contacting the phage display library with a sample containing antibody, immunoselection of phage displaying antigen bound by antibody which complex is immobilized by a substrate, and sequencing the immunoselected phage for determining the antigenic epitope recognized by antibody in the sample.

FIG. 2 is a series of histograms showing range of cDNA insert sizes from different phage libraries produced based on tissue or cell source (e.g., Hep-2, fetal astrocytes, and brain white matter) of the originating mRNA. Mean cDNA insert sizes for each library are also shown.

FIG. 3A is a Venn diagram showing the analysis of genes differentially expressed by the cells of the original source of mRNA (Hep-2, fetal astrocytes, and brain white matter (“brain”)) prior to phage display library production.

FIG. 3B is a Venn diagram showing the analysis of proteins encoded by genes differentially expressed by the cells of the original source of mRNA (Hep-2, fetal astrocytes), and brain white matter (“brain”) after phage display library production, pooling of the phage display libraries produced, and immunoselection with serum from either healthy individuals (“Healthy”), serum from individuals with systemic lupus erythematosus (“SLE”) or serum from individuals with Neuromyelitis optica (NMO). Negative “Control” samples were where CD20 monoclonal antibody or no antibody was used in the phage selection assays.

FIG. 4 is a heatmap illustrating antibody signatures for 5 individuals having NMO, showing the top 30 gene-encoded proteins containing antigenic epitopes immunoselected for with antibodies contained in samples from these 5 individuals. Intensity of color reflects the relative number of deep-sequencing counts for each gene observed for each sample expressed at a logarithmic scale.

FIG. 5A is a heatmap illustrating antibody signatures for 15 individuals having SLE, as compared to antibody signatures for 23 healthy individuals (“Healthy”) showing the top 40 gene-encoded proteins containing antigenic epitopes immunoselected for by antibodies contained in samples from the 15 individuals with SLE. Intensity of color reflects the relative number of deep-sequencing counts for each gene-encoded protein observed for each sample expressed at a logarithmic scale.

FIG. 5B is a heatmap illustrating antibody signatures for 15 individuals having SLE, as compared to antibody signatures for 23 healthy individuals (“Healthy”) shown in FIG. 5A wherein autoantigens known to be associated with SLE are identified. Intensity of color reflects the relative number of deep-sequencing counts for each gene-encoded protein observed for each sample expressed at a logarithmic scale.

FIG. 6A is a heatmap illustrating antibody signatures for plasma samples from 5 individuals with NMO, plasma from 5 individuals with SLE (“Lupus”) and plasma from 5 healthy individuals (“Healthy”) relative to 30 gene products selected most robustly by antibodies contained in samples from individuals with NMO. Also shown are 6 negative control assays (“Control”). Three control assays used a chimeric anti-human CD20 monoclonal antibody. Since CD20 is not expressed by the cell types used for library construction, this controlled for the selection of phage that would non-specifically bind to components of the test system such as plasticware, paramagnetic beads, blocking proteins, or antibody regions not involved in antigen recognition. The CD20 antibody concentration was matched to serum IgG levels (10 mg/ml). Three other controls included library phage assayed without antibody present to control for background phage binding, and for fast growing and overabundant phage clones within the libraries during the immunoselection assays. Intensity of color reflects the relative number of deep-sequencing counts for each gene-encoded protein observed for each sample expressed at a logarithmic scale.

FIG. 6B is a quantitative graph illustrating antibody signatures for plasma from 5 individuals with NMO, plasma from 5 individuals with SLE (“Lupus”) and plasma from 5 healthy individuals (“Healthy”) relative to 30 gene products selected most robustly by antibodies contained in samples from individuals with NMO. Read counts reflect the number of deep-sequencing counts for each gene-encoded protein observed for each sample as in FIG. 6A as expressed at a logarithmic scale.

FIG. 7A is a heatmap illustrating the reproducibility of generating an antibody signature using antibodies from the same sample of an individual with NMO, but from 4 independent experiments (“1”, “1A”, “1B”, and “1C”) with sample 1 sequenced at 20-fold higher depth than 1A, 1B, and 1C, and as compared to the antibody signatures from samples of 4 other individuals with NMO (“2”, “3”, “4”, “5”) relative to 30 gene products selected most robustly by antibodies contained in the sample from individual “1” with NMO. Intensity of color reflects the relative number of deep-sequencing counts for each gene-encoded protein observed for each sample expressed at a logarithmic scale.

FIG. 7B are scatter plots illustrating the reproducibility of generating antibody signatures from the same serum samples of three individuals; one healthy (sample 153), one with SLE (sample 107), and one with NMO (sample 202). Autoantigen counts were obtained from two independent serum selection experiments and were independently deep sequenced as shown on each axis, with each dot representing a unique gene-encoded protein with total counts >100 on both log-scale axes. The diagonal line indicates the correlation between experiments for 100 proteins with the highest total counts after sequencing. Proteins with counts below 1000 in experiment 2 deviate from the correlation trend because of sequencing depth differences in the two sequencing runs.

FIG. 7C is a heatmap illustrating the reproducibility of generating an antibody signature from the same serum samples of three individuals as in FIG. 7B. Antibody signatures for each individual were compared to the antibody signatures of individuals randomly selected from the same cohort (163, 119, and 211). Autoantigen counts were sorted based on the count abundance in experiment #1. The autoantigen ranking shows the top 100 of all gene-encoded proteins containing antigenic epitopes immunoselected for by serum antibodies as sorted on samples 153, 107 and 202, with the same antigens represented similarly across rows within each of the three data panels. Thereby, autoantigen rankings were different between each of the three data panels.

FIG. 8 is a graph illustrating clonal enrichment of phage expressing human cDNAs by antibodies specific to five human proteins (AB12, CALD1, UBA1, NONO, PCNA) and a control antibody (ITGB1) during three rounds of phage immunoselection (Ab Mix, round I-III) relative to the unselected human antigen phage display library (No Selection, round I). Commercial rabbit antibodies elicited by immunizations with 50 amino acid regions of each protein (ABI2 351-401 aa, CALD1 675-725 aa, UBA1 800-850 aa, NONO 350-400 aa, PCNA 225-C-term aa, ITGB1, 650-700 aa) were spiked into a well-characterized human monoclonal antibody sample that was used to select phage. Data represent normalized deep-sequencing counts attributable to each protein after each round of selection.

FIG. 9 is a heatmap illustrating antibody signatures for 15 individuals having SLE, as compared to antibody signatures for 23 healthy individuals (“Healthy”). The autoantigen ranking shows the top 50 of all gene-encoded proteins containing antigenic epitopes immunoselected for by antibodies contained in each serum sample (columns). SLE-specific autoantigens (rows) were ranked during bioinformatics analysis based on their level of statistical significance (p-value) relative to the matched cohort of healthy individuals. Intensity of color reflects the relative number of deep-sequencing counts for each gene-encoded protein observed for each sample expressed at a logarithmic scale. In the bottom panel, autoantigens known to be associated with SLE were identified. The common name of each autoantigen is shown, followed by its autoantigen ranking as shown in the top panel. In cases where multiple rows have the same autoantigen name, each row represents a distinct subunit or isoform of the protein.

FIG. 10 shows the validation of Antigenome Signatures using Antinuclear Autoantibody (ANA) serum standards distributed through the Centers for Disease Control. Each column represents an individual ANA standard serum, SLE or healthy individual serum, or background control sample. Known autoantigen target specificities for each ANA standard sera is indicated below the heatmap columns. Each row indicates known ANA target autoantigens. Intensity of color reflects the relative number of deep-sequencing counts for each autoantigen observed for each sample expressed at a logarithmic scale.

FIG. 11 is a heatmap illustrating the individual ranking of autoantigen specificities for six individual ANA Standard Sera. Identified autoantigens were ranked from the most abundant (highest counts) to the least abundant for each ANA serum with the twenty autoantigens with the highest counts shown. Black circles indicate the ranked position of the autoantigens to which the ANA sera has known specificity.

FIG. 12 is a comparison of results obtained using the current antigen selection assay and a diagnostic ELISA test for quantifying SSB/La specific autoantibodies in patient's sera. Thirty sera with a range of reactivities were tested in both assays. Four sera with ELISA values >30 were considered positive based on the ELISA manufacturer's criteria. The best fitting line representing these four positive sera was determined using the linear least squares fitting technique.

FIG. 13 is a compendium of the unique SSB/La protein fragments present within the pooled human antigen display library utilized for serum sample screening. The dominant SSB domain fragment selected by SLE patient's serum autoantibodies is indicated as a dashed line. The domain structure of SSB/La is shown at the bottom of the figure. NLS denotes the nuclear localization signal.

FIGS. 14A and 14B shows dominant SSB/La protein fragments from the pooled human antigen display library that were enriched following selection using SLE patient's sera 109 (FIG. 14B) and 119 (FIG. 14A). Y-axis values represent the fold-increase in fragment counts after serum selection relative to the fragment counts present within the unselected antigen display libraries.

### DETAILED DESCRIPTION OF THE INVENTION

One microliter of human serum or plasma from an average adult, contains approximately 5.8×10¹⁶ antibody molecules, including antibodies of the IgM, IgG, IgA and IgE classes. Provided herein are methods of making phage display libraries that contain enormous diversity of inserts to enable the measurement of antibody-binding epitopes on expressed proteins (including fragments thereof), whether from the human genome, the microbiome, infectious agents, or the environment. The phage libraries are constructed such that in-frame, coding region transcription units are expressed in the majority or substantially all of the recombinant phage, and contain an enormous diversity of protein epitopes that are predominantly domain-sized protein fragments with secondary and tertiary structure. Correct orientation and length of DNA fragments aid to preserve the reading frame of a corresponding native peptide and reading frame of the phage protein fused at the C-terminus. Also provided is effective, accurate, and efficient ways of measuring the interactions between antibodies in the sample and phage expressing linear and conformational antigen epitopes expressed and displayed by such diverse phage display libraries. The methods utilize identification of antigen in solution, thereby preserving the secondary and tertiary domain structure of the protein as compared to assays that depend on the attachment or capture of the antigen or peptides on a solid surface.

Definitions—While the following terms are believed to be well understood by one of ordinary skill in the art of biotechnology, the following definitions are set forth to facilitate explanation of the invention.

The term “antibody signature” is used herein to mean the spectrum of antigens or antigenic epitopes recognized by the antibodies derived from a biological sample, as determined by the antigenic display system provided herein. The term antigen display system refers to the antigen display library and may include other reagents needed to use the system. The spectrum of antigens identified by antibody binding may be used to generate a pattern or dataset illustrating a relationship between the antigenic epitopes, expressed by an antigen display library, that are recognized by antibodies derived from the sample. An analytical approach using bioinformatics is used to analyze the data generated from independent experiments so as to consistently and reproducibly compare antibody signatures between individuals, within the same individual over time, between different bodily fluids, and between samples from individuals in different categories of disease processes. The relationship may be expressed in a pattern (“signature”), such as generated by one or more commercially available computer algorithms or software, and if desired, may further be graphically expressed in visual form, such as a Venn diagram, heat map, data clustering map, quantitative graph, volcano plot, scatter plot, dendrogram, data cluster, principal component analysis, gene network analysis, GSEA plot, and other methods known to those with skill in the art. Parameters useful in generating an antibody signature include, but are not limited to, the level of antibodies to a specific antigen, diversity of antigens (e.g., differing by one or more of genetic sequence or occurrence in a disease process or from a healthy individual), epitope mapping of antibody binding sites within proteins, diversity of antigens shared between disease cohorts, numbers of antigens correlated with a disease, disease process, therapeutic outcome or diagnostic feature. An antibody signature may be compared with a reference or control antibody signature (e.g., from analysis of a sample or set of samples from an unaffected, normal, or healthy individual(s)). Additionally, a reference antibody signature may be a signature pattern established from samples obtained from individuals suspected of having or known to have the same disease process. Antibody signatures may also reveal individuals who may be responsive or non-responsive to a therapy of interest, and thereby such signatures may be useful as a factor to consider in treatment decisions. An algorithm that combines the results of the antibody specificity for antigens as a dataset, can be used to generate an antibody signature. The dataset comprises quantitative data reflecting or quantifying the presence of antibodies from a sample analyzed, detecting a plurality of antigens or antigenic epitopes from the antigenic display library. The plurality of antigens or antigenic epitopes recognized by antibody and used in generating the antibody signature may range from 10 to 100 to 20,000 to 5,000,000 or more antigens or epitopes thereof. In order to identify profiles that are indicative of a disease process or of diagnostic and/or therapeutic value, a statistical test is used to provide a confidence level for a change in the expression or amount of detected antibodies to antigens between a test antibody signature (e.g., produced from one or more samples from one or more individuals suspected of having or known to have a disease process) and a control or reference antibody signature (e.g., produced from one or more samples from one or more persons known not to have the disease process) to be considered significant using statistical analyses standard in the art. A test antibody signature is considered to be different from a control or reference antibody signature where at least 1, at least 3, usually at least 5, at least 10, at least 15 or more of the antigens, or epitopes thereof, of the test antibody signature are statistically different (at a predefined level of significance) in a parameter (e.g., selected from one or more of level of occurrence, expression or detection) as compared to the control or reference antibody signature.

The term “antigen” is used herein to mean, when referring to detection by an antibody, an antigen or the portion of an antigen (antigenic epitope) that makes contact with an antibody having binding specificity for the antigen. Self-antigen or autoantigen is an antigen that is normally present in the body of an individual to which antibodies having binding specificity therefor are not detectable or are found at significantly lower levels in the absence of a disease process, but as a result of a disease process to which antibodies having binding specificity therefor are induced. An autoantibody refers to an antibody having binding specificity for an autoantigen. An antigen can stimulate the production of antibody, and can be bound by antibody specific for the antigen (i.e., an antibody can specifically bind an antigen for which it has binding specificity). Antigens may be comprised of a substance comprising one or more of protein, peptide, lipid, phospholipid, carbohydrate, nucleic acid, and small molecule (organic or inorganic). Antigens may include: a substance foreign to the human body, viral antigens, bacterial antigens, parasite antigens, tumor antigens, toxin antigens, fungal antigens, self-antigens, altered self-antigens (self-antigens that are altered or modified as the result of a disease process), modified antigens (misfolded or oxidized or with altered glycosylation or overexpression or mutated, as a result of a disease process and as compared to the antigen in a healthy individual or in the absence of a disease process). Illustrated in Table 1 are some known autoantigens for human diseases including systemic lupus erythematosus (SLE), Neuromyelitis optica (NMO), rheumatoid arthritis (RA), autoimmune blistering dermatoses (ABD), diabetes (Type 1), multiple sclerosis (MS), Sjögren's syndrome, polymyositis, and celiac disease.

TABLE 1

| Disease | Autoantigen |
|---|---|
| SLE | proteins complexed to Uridine-rich (u) RNAs (U1, U2, U4, U5 SnRNP) or to |
| | small cytoplasmic RNAs (hY-RNAs), histone proteins (H1, H2A, H2B, H3, |
| | H4), proteins associated with U1 RNP (70 Kd, A & C proteins), |
| | phosphorylated ribosomal proteins (P0, P1, P2), topoisomerase 1 |
| NMO | Aquaporin-4, myelin oligodendrocyte glycoprotein (MOG) |
| RA | filaggrin, keratin, Sa, Hsp65, Hsp90, DnaJ, BiP, hnRNPA2 (Ra33), annexin |
| | V, calpastatin, type II collagen, glucose-6-phosphate isomerase (GPI), |
| | elongation factor, human cartilage gp39, citrullinated vimentin, type II |
| | collagen, fibrinogen, alpha enolase, carbamylated antigens (CarP), |
| | peptidyl arginine deiminase type 4 (PAD4), BRAF (v raf murine sarcoma |
| | viral oncogene homologue B1), fibronectin, immunoglobulin binding |
| | protein (BiP). |
| ABD | DSG-3, DSG-1, desmoplakin I, envoplakin, periplakin, desmocollin 3 |
| Diabetes | Insulin, IAA, ICA2, GAD65, Hsp60 |
| MS | Myelin proteins [Myelin oligodendrocyte glycoprotein (MOG), myelin |
| | basic protein (MBP), proteolipid protein (PLP), myelin-associated |
| | glycoprotein (MAG), phosphatidylcholine, galactocerebroside (GalC) |
| Sjögren's | Ro, La, SP-1, CA6 and PSP |
| Polymyositis | aminoacyl-transfer ribonucleic acid (tRNA) synthetases, nuclear Mi-2 |
| | protein, components of the signal-recognition particle (SRP), PM/Scl |
| | nucleolar antigen (75&100), the nuclear Ku antigen, the small nuclear |
| | ribonucleoproteins (snRNP), and the cytoplasmic ribonucleoproteins |
| | (RORNP), TIF1-γ, MDA5, NXP2, SAE, and HMGCR |
| Celiac disease | Tissue transglutaminase (TG2, TG3 and TG6), deaminated gliadin, R1 |
| | type reticulin |

The term “antigen display library” is used herein to mean a phage-based library of recombinant phage displaying on their surface antigens derived from various sources including, without limitation, cDNA reverse transcribed from mRNA isolated from one or more cell types, cells from one or more tissue types (disease-specific or healthy tissues), cells from one or more organs, or a pool of Ff phage libraries (combination thereof). The cell types used may be from a mammal. The DNA inserts may also be synthetically produced based on protein-coding regions of DNA from any known cell or organism. The DNA inserts are selected to comprise a length selected from between about 150 and 900 nucleotides and are selected for in frame expression as part of a gene. The diversity of peptides (which may be antigenic epitopes) encoded by the DNA inserted in the phage library comprising the antigen display library is estimated to be greater than 1×10⁶.

The antigen display libraries in the examples were generated from human cells such as HEp-2 cells or isolated astrocytes. The antigen display libraries can also be generated from tissue types such as the white brain matter used in the examples. Those skilled in the art will understand that many other tissue types could be used and how to select cells or tissues to assess various disease states. Antigen display libraries can also be generated from yeast and other small, replicating organisms.

Prior to cloning the DNA into the phage vector in constructing the phage library, the DNA is selected for a size ranging from about 150 nucleotides to about 900 nucleotides in length to facilitate the detection of sequences that encode linear epitopes and conformational epitopes. In alternative embodiments the DNA may be size selected for a narrower range of sizes such as 200 to 800 nucleotides, 225 to 700 nucleotides, 250 to 600 nucleotides or other ranges there between such as 200 to 600 which was used in the examples. Suitably the size of the DNA insert is larger than 150, 180, 210, 240, 270, or 300 nucleotides. Suitably, the DNA insert is less than 900, 870, 840, 810, 780, 750, 720, 690, 660, 630 or 600 nucleotides. Any range between these indicated numbers of nucleotides as an average insert size is useful and may vary depending on the specific application. The size selection of the DNA segments allows for cloning of domain sized fragments of proteins that are likely to produce appropriate secondary and tertiary structure when inserted in a phage coat protein and thus preserve conformational epitopes as well as linear epitopes. The DNA may be made in a way that allows for overlapping peptide fragments of the protein to be generated because some fragments will be more likely to produce the correct conformation than others. Although the selection procedure selects for a particular size range, it will be appreciated that some DNA inserts may have a size that falls outside that range (i.e., below 150 nucleotides or above 900 nucleotides). The DNA inserts, as a whole, however may have an average length within the ranges described herein.

The size-selected DNA is also selected for in-frame DNA fragments by directional molecular cloning into a plasmid containing a selectable marker to allow selection of positively transformed cells so that only insert-encoded polypeptides that were in-frame with a selectable marker (e.g., plasmid β-lactamase gene (ampicillin resistance), aminoglycoside phosphotransferase (neo), chloramphenicol acetyltransferase (cat), or mutated enoyl ACP reductase (mfabl) genes, neomycin- or other antibiotic resistance gene) at the 3′ end of the DNA insert would be expanded during plasmid library amplification. The use of cDNA is one way to aid in this selection. Other selectable markers useful for such purpose include, but are not limited to antibiotic resistance genes, such as tetracycline, fluorescent markers such as GFP, eGFP, YFP, CFP, BFP, and RdFP. As a result, this antigen display library, and the method of constructing it, requires the phage to express protein domains that have to be in-frame, translatable, and able to be expressed. Therefore, it is important that empty phage are not detectably generated, which allows for the generation of antigen display libraries with high domain diversity as compared to other antigen display libraries described in the art.

The phage used in the antigenic display libraries in the Examples comprises Ff phage (filamentous phage that infect gram negative bacteria bearing the F episome) including but not limited to f1, fd, and M13. Related Ike phage, T4, T7 and If1 phage may also be used. In one aspect, Ff phage used to produce the antigen display library comprises M13 bacteriophage. In one aspect, M13 phage was used to express human cDNA-encoded proteins at low- or high-densities on the phage surface, which were generated using two M13 filamentous phage systems with N-terminal fusions to the coat proteins pill or pVIII. The low density antigen display libraries expressed human cDNA-encoded polypeptides fused at the N-terminus of the pill coat protein that is present at 5 copies per virion. This pill protein phage display system utilized the pSEX81 phagemid where 1 to 5 pill-human cDNA-encoded fusion protein molecules that don't interfere with phage infectivity can be expressed on the surface of each phage particle. Given the low density of fusion proteins per phage, this system is advantageous for examining high affinity protein:protein interactions. By contrast, high-density antigen display libraries were generated using the pG8SAET phagemid, where human polypeptides produced by recombinant phage were fused to the N-terminus of the major M13 coat protein pVIII. There are at least approximately 2,700 copies of the pVIII protein expressed per phage virion. Since bacteria are superinfected with a helper phage that encodes for a wild type pVIII, pVIII coat protein is produced as both a native protein and a cDNA insert fusion protein in this system, enabling the production of phage even when coat protein assembly may be limited by the structure of the pVIII-human antigen fusion protein. Approximately 10% of the expressed virion surface pVIII can be reliably fused to peptides or proteins, allowing for the expression of over 270 fusion proteins per viral particle. Thereby, the pVIII expression system enables both high and low affinity antibody:antigen interactions.

The terms “binding specificity”, “recognized” and “bound” when referring to the interaction between an antigen and antibody, refer to a chemical interaction between chemical molecules (e.g., amino acids, carbohydrates or lipids) of an antigen and chemical molecules (e.g., amino acids) comprising the binding site of the antibody which is induced by the antigen. These interactions are non-covalent and may include all forms of non-covalent interactions.

The terms “biological sample” or “sample” are used herein and interchangeably refer to samples obtained from one or more of tissues or fluids of an individual. Tissues may be obtained from an individual by biopsy, and then processed using methods know in the art for providing a sample comprising antibodies. Sources of body fluids that comprise antibody or may be analyzed for the presence of antibodies, includes but is not limited to, whole blood, fractions of blood (e.g., serum, plasma), saliva, exudate, synovial fluid, lymph, cerebrospinal fluid, aspirates, breast milk, urine, and the like. A biological fluid, if desired, may be further processed using methods know in the art for providing a sample comprising antibodies (e.g., fractionation, purification, concentration, dilution, etc.).

The term “disease process” is used herein to mean any deviation from normal processes that contribute to the health of an individual. The disease process may be a condition, syndrome, disorder, dysregulation, or disease, and include but is not limited to, cancer, inflammation, autoimmunity, neurologic, behavioral, psychiatric, metabolic, an imbalance of one or more chemical mediators, and the like. The disease process may be an immune-related disease. Many immune-related diseases are known in the art, and have been extensively studied. Immune-related diseases include immune-mediated inflammatory diseases (such as arthritis (e.g., rheumatoid arthritis, psoriatic arthritis), immune-mediated diseases of an organ or body system (immune-related kidney disease, hepatobiliary diseases, inflammatory bowel disease, psoriasis, allergy, autoimmunity, and asthma); non-immune-mediated inflammatory diseases; immunodeficiency diseases; fibrosis; diabetes; non-alcoholic fatty liver disease; and cancer. Autoimmune diseases and autoantibody-associated syndromes are known in the art to include, but are not limited to, acute disseminated encephalomyelitis (ADEM), Addison's disease, agammaglobulinemia, alopecia areata, amyloidosis, ankylosing spondylitis, anti-GBM/anti-TBM nephritis, anti-phospholipid syndrome, autoimmune encephalitis, autoimmune hepatitis, autoimmune inner ear disease, axonal & neuronal neuropathy (AMAN), autoimmune polyendocrinopathy, Behcet's disease, bullous pemphigoid, Castleman disease, celiac disease, cerebellar syndrome, Chagas disease, chronic fatigue syndrome, chronic inflammatory demyelinating polyneuropathy (CIDP), chronic recurrent multifocal osteomyelitis (CRMO), Churg-Strauss syndrome, cicatricial pemphigoid/benign mucosal pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), diabetes incipidus, discoid lupus, Dressler's syndrome, drug-induced erythematosus, Duhring's dermatitis herpetiformis, endometriosis, eosinophilic esophagitis (EoE), epidermolysis bullosa, eosinophilic fasciitis, erythema nodosum, essential mixed cryoglobulinemia, evans syndrome, fibromyalgia, fibrosing alveolitis, giant cell arteritis (temporal arteritis), funicular myelosis, giant cell myocarditis, glomerulonephritis, Goodpasture's syndrome, granulomatosis with polyangiitis, Graves' disease, Guillain-Barre syndrome, habitual abortions, Hashimoto's thyroiditis, hemolytic anemia, Henoch-Schonlein purpura (HSP), heparin-induced thrombocytopenia, Herpes gestationis or pemphigoid gestationis (PG), hypogammalglobulinemia, IgA nephropathy, IgG4-related sclerosing disease, idiopathic thrombocytopenic purpura (ITP), idiopathic urticaria, inclusion body myositis (IBM), inflammatory bowel disease, interstitial cystitis (IC), juvenile idiopathic arthritis, juvenile diabetes (Type 1 diabetes), juvenile myositis (JM), Kawasaki disease, Lambert-Eaton syndrome, laminin γ1 pemphigoid, leukocytoclastic vasculitis, lichen planus, lichen sclerosus, ligneous conjunctivitis, linear IgA disease (LAD), systemic lupus erythematosus (SLE), lyme disease, Meniere's disease, microscopic polyangiitis (MPA), Miller-Fisher syndrome, mixed connective tissue disease (MCTD), Mooren's ulcer, Mucha-Habermann disease, mucous membrane pemphigoid, multifocal motor neuropathy, multiple sclerosis (MS), myasthenia gravis, myocarditis, myositis, narcolepsy, neonatal idiopathic thrombocytopenic purpura, neonatal lupus erythematosus, neuromyelitis optica, neuromyotonia, neutropenia, ocular cicatricial pemphigoid, opsoclonus myoclonus, optic neuritis, palindromic rheumatism (PR), PANDAS (Pediatric Autoimmune Neuropsychiatric Disorders Associated with Streptococcus), parainfectious enzephalitis, paraneoplastic autoimmunity, pandysautonomia, paraneoplastic cerebellar degeneration (PCD), paroxysmal nocturnal hemoglobinuria (PNH), Parry Romberg syndrome, Pars planitis (peripheral uveitis), Parsonnage-Turner syndrome, pemphigus vulgaris, pemphigus foliaceus, pemphigoid gestationis, peripheral neuropathy, perivenous encephalomyelitis, pernicious anemia (PA), POEMS syndrome (polyneuropathy, organomegaly, endocrinopathy, monoclonal gammopathy, skin changes), polyarteritis nodosa, poly-dermatomyositis, polymyalgia rheumatica, polymyositis, postmyocardial infarction syndrome, primary biliary cirrhosis, postpericardiotomy syndrome, primary biliary cirrhosis, primary sclerosing cholangitis, progesterone dermatitis, psoriasis, psoriatic arthritis, psychosis, pure red cell aplasia (PRCA), pyoderma gangrenosum, Raynaud's phenomenon, reactive arthritis, reflex sympathetic dystrophy, Reiter's syndrome, recurrent optic neuritis, relapsing polychondritis, restless legs syndrome (RLS), retinopathy, retroperitoneal fibrosis, rheumatic fever, rheumatoid arthritis (RA), sarcoidosis, Schmidt syndrome, scleritis, scleroderma, sensory neuropathy, Sharp syndrome (MCTD), Sjogren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes (mellitus), ulcerative colitis (UC), undifferentiated connective tissue disease (UCTD), uveitis, vasculitis, vitiligo, and Wegener's granulomatosis (now termed Granulomatosis with Polyangiitis (GPA).

The term “substrate” is used herein to mean a solid support or matrix to which antibody is immobilized (either prior to contacting with antigen or as a part of a complex of antibody and antigen) which can then be used to capture and aid in subsequently identifying phage-expressed antigens recognized by the antibody. The substrate may include an affinity substrate capable of specifically binding antibodies or specifically binding a class of antibodies. For example beads may be used as a substrate and may be coated with an affinity substrate such as protein A or an antibody specific for at least one of IgG, IgA, IgM, IgD or IgE.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The present invention will be described in the following examples, which are illustrative in nature.

### EXAMPLES

### Example 1

### Production of Antigen Display Library

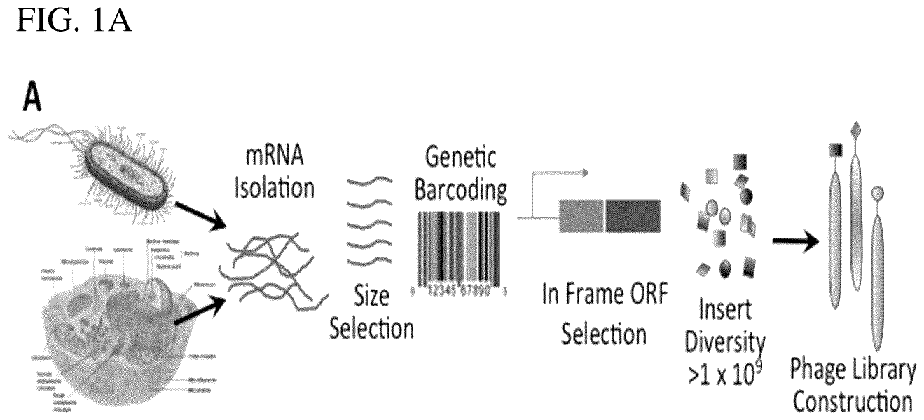

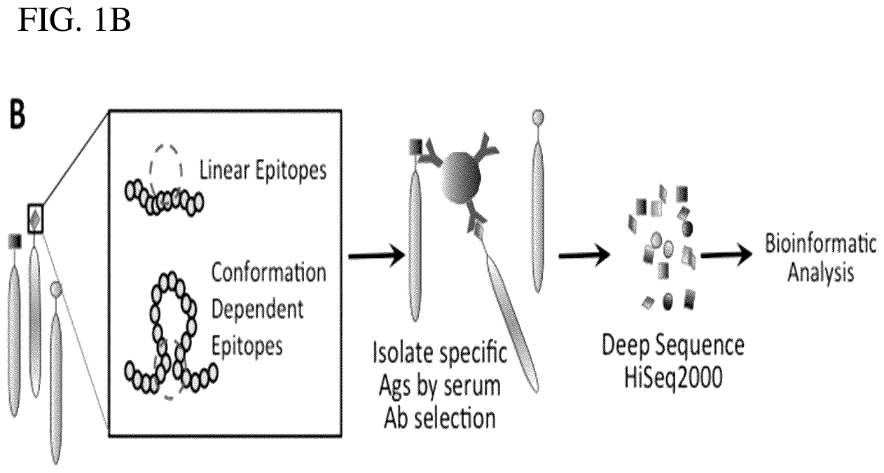

In one aspect, a method of producing a phage display library for expression and presentation of linear epitopes and conformational epitopes, and its use to characterize antibody responses to the antigens, the method comprises (a) converting mRNA, from a cell type or tissue type, to cDNA using primers with adapters that allow for subsequent directional cloning into a vector; (b) size selecting the cDNA by selecting cDNA in a size range of from about 150 bp to about 900 bp; (c) directionally cloning of the size-selected cDNA as inserts into a plasmid vector comprising a selectable marker (e.g., antibiotic resistance gene, or reporter gene), to allow selection of positively transformed cells when the inserts are in-frame with the selectable marker to facilitate expression of the selectable marker, in forming recombinant vector; (d) transforming recombinant vector into cells; (e) selecting cells carrying recombinant vector with in-frame inserts by identifying cells expressing the selectable marker; (f) purifying plasmids with in-frame inserts from the selected cells; and (g) subcloning the inserts into an Ff phage vector in forming recombinant phage; to produce a phage display library. FIG. 1A is a schematic diagram summarizing production of the phage display library for the expression and presentation of linear epitopes and conformational epitopes, and its use to identify and characterize antibody responses to the antigens. The size-selected, directionally clonable cDNA insert may further comprise (before subcloning into a vector, or as part of the vector sequence which then is later cleaved to become part of the cDNA insert) a unique barcode comprised of contiguous nucleotides ranging from about 5 to about 20 nucleotides which may be used to identify inserts from a specific phage library in a pool of phage libraries.

In one aspect, mRNA isolated from one or more cell type or tissue type of human origin is used for the creation of phage libraries. In one aspect, more than one phage library is created, with each phage library derived from mRNA from a different cell type or tissue type as compared to that used for creation of the other phage libraries created. This allows for maximum diversity for each individual phage library during creation, while allowing for pooling of phage libraries for expanding the number of antigenic epitopes displayed for immunoselection using a biological sample containing antibodies. In an illustrative example, total RNA was obtained from HEp-2 cells, astrocytes, and normal appearing white brain matter. Total RNA was purified using standard reagents (e.g., TRIzol reagent) and methods known in the art. mRNA (Poly-A+RNA) was purified from total RNA using a commercially available magnetic mRNA isolation kit. cDNA was synthesized and then size-selected for cloning into phage vector. Poly-A′ RNA was converted to cDNA using a random hexamer primer with an adapter that encodes a Notl endonuclease restriction site (5′-GCGGCCGCAACNNNNNNNNN-3′; where N is random, being A, T, G and C within the mixture; SEQ ID NO:1), which is required for subsequent downstream directional cloning. A second strand cDNA was then generated using a random hexamer primer (5′-TGGCCGCCGAGAACNNNNNNNNN-3′; SEQ ID NO:2) with an encoded NcoI site and the Klenow fragment (3′->5′ exo -) that lacks 3′->5′ exonuclease activity. Double stranded DNA was purified using a commercially available kit according to the manufacturer's instructions.

The cDNA generated above was amplified by polymerase chain reaction (PCR) using a forward primer comprising SEQ ID NO:3 (5′-GCTGGTGGTGCCGTTCTATAGCCATAGCACCATGGCCGCCGAGAAC-3′) and reverse primer comprising SEQ ID NO: 4 (5′-TTTTACTTTCACCAGCGTTTCTGGGTGAGCTGCAGCGG CCGCAAC-3′) for 13 cycles using the following settings: 94° C. for 20 seconds, 62° C. for 10 seconds, and 72° C. for 45 seconds. After amplification, cDNA fragments of 200 to 600 bp were size selected using solid phase reversible immobilization magnetic beads. After binding cDNA, the beads were pelleted in a magnetic field, washed twice with 80% ethanol, and dried before the bound cDNA was eluted in water. The size-selected cDNA was then assessed for size by gel electrophoresis and quantified using a commercially available kit highly selective for quantitating cDNA.

The size-selected cDNA was directionally inserted into linearized plasmid vector containing a selectable marker. In this example, the vector comprised the pBADSelect vector (engineered from a pBAD-family vector by deleting the nucleotides between the NcoI site within the multiple cloning site and the nucleotides encoding the 23rd amino acid of the ampicillin resistance gene with a small stuffer insert inserted to allow for the introduction of a Notl site within the ampicillin resistance gene). The pBADSelect vector was linearized using Notl-HF and NcoI-HF endonucleases and gel purified, followed by ligation with the cDNA inserts to create recombinant plasmid comprising a cDNA plasmid pool. To preserve maximal diversity within the cDNA plasmid pool prior to bacterial transformations and to minimize biased clonal amplifications, cDNA insert-containing plasmids were amplified using phi29 DNA polymerase through a rolling circle amplification procedure using 3′ exonuclease-resistant random heptamer primers and dNTPs under optimized conditions. The polymerase was inactivated by incubation at 65° C. for 10 minutes. Phi29 amplification resulted in long linear concatenated DNA strands that were then digested with Notl-HF restriction enzyme according the manufacturer's recommendations, prior to circularization using T4 DNA ligase according to the manufacturer's recommendations. The ligase was inactivated by incubation at 65° C. for 15 minutes. The DNA was then concentrated using DNA concentrators per the manufacturer's instructions and eluted in water. The resultant recombinant plasmids were used to transform bacteria, and then the transformants were selected for expression of a selectable marker for identifying transformants containing plasmid with inserts cloned in-frame with the gene encoding the selectable marker.

To promote high transformation efficiencies and high library diversity, commercially available E. coli electrocompetent cells were electroporated with 1.5 μg of the amplified cDNA insert-containing plasmids using methods known in the art. The electroporated cells were diluted to 2 ml with microbial growth medium used for the transformation of competent cells (SOC media), pooled and cultured at 37° C. for 35 minutes. The transformed bacteria were then plated using sterile glass beads onto 15 cm 1.5% agar LB (Luria broth) plates containing 0.2% L-arabinose. Half of the plates contained carbenicillin at 30 μg/ml, and half contained carbenicillin at 75 μg/ml to select for transformed bacteria. The lower concentration of carbenicillin was used to maintain bacteria that were transformed with plasmids containing cDNA inserts that impede translation of the in-frame β-lactamase selection marker, thereby maintaining the overall diversity of the library. Bacteria containing plasmids lacking cDNA inserts, or plasmids with cDNA inserts that were out-of-frame with, or that contained stop codons are unable to produce in-frame, β-lactamase and thereby remain carbenicillin sensitive. The seeded culture plates were incubated at 30° C. for 22 hours, with bacterial colonies harvested from the agar surface by scraping. Plasmid DNA was purified separately from bacteria (7.5×10¹⁰) cultured at each antibiotic concentration using a commercially available plasmid midiprep kit according to the manufacturer's instructions.

The size-selected, directionally cloned, in-frame, amplified cDNA inserts (“human cDNA inserts”) were removed from the plasmid vector and then cloned into the desired phagemid vector as follows. Purified pBADSelect plasmid containing human cDNA inserts (300 ng) was used as a template for generating cDNA amplicons that were inserted into pSEX81 or pG8SAET phagemid plasmids. Human cDNA inserts for insertion into the pSEX81 cloning vector were generated by PCR using a forward primer comprising SEQ ID NO: 5 (5′-TAAACAACTTTCAACAGTTTCAGCTCTGATATCTTTGGATCCAGCGGCCGCAAC-3′), a reverse primer comprising SEQ ID NO:6 (5′-CCGCTGGCTTGCTGCTGCTGGCAGCTCAGCCGGCCATGG CCGCCGAGAAC-3′), and DNA Polymerase. PCR amplification was carried out for 11 cycles; 94° C. for 20 seconds, 47° C. for 10 seconds, and 72° C. for 45 seconds. Human cDNA inserts for insertion into the pG8SAET cloning vector were generated by PCR using a forward primer comprising SEQ ID NO: 7 (5′-GTTCCAGTGGGTCCGGATACGGCACCGGCGCACCGGCGGCCGCAAC-3′) a reverse primer comprising SEQ ID NO:8 (5′-TGGCGTAACACCTGCTGCAAATGCTGCGCAACACGCCATGGCCGCCGAGAAC-3′), and DNA Polymerase. PCR amplification was carried out for 12 cycles; 94° C. for 20 seconds, 53° C. for 15 seconds, and 72° C. for 45 seconds. The pBADSelect plasmid DNA template was removed from the reaction mixtures after PCR amplification by digestion with DnpI endonuclease, which cleaves methylated DNA, for 1 hour at 37° C. The DNA amplicons were then purified by phenol/chloroform extraction with the subsequent isolation of 200-600 bp DNA fragments (human cDNA inserts) using solid phase reversible immobilization magnetic beads as described above. The DNA amplicons were quantified using a commercially available kit highly selective for quantitating cDNA, and combined at equimolar ratios.

The DNA amplicons were subcloned into either the pSEX81 phagemid or pG8SAET phagemid for the generation of either low density or high-density phage display libraries, respectively. Linearized pSEX81 or pG8SAET cloning vectors were generated by PCR using empty phagemids as templates and two pairs of primers: for pSex81, a forward primer comprising SEQ ID NO:9 (5′-CGGCCGCTGGATCCAAA G-3′) and a reverse primer comprising SEQ ID NO:10 (5′-CCATGGCCGGCTGAGCTG-3′); and for pG8SAET, a forward primer comprising SEQ ID NO:11 (5′-GCGGCCGCCGGTGCGCCGGTGCC-3′) and a reverse primer comprising SEQ ID NO:12 (5′-CCATGGCGTGTTGCGCAGCATTTGC-3′). PCR amplification was performed for 26 cycles using DNA Polymerase under the following conditions: for pG8SAET, 94° C. for 15 seconds, 65° C. for 15 seconds, 70° C. for 4 minutes; and for pSex81, 94° C. for 15 seconds, 65° C. for 15 seconds, 70° C. for 5 minutes. After PCR amplification, the template plasmid was removed by digestion with DpnI endonuclease. The linearized vector amplicons were purified by gel electrophoresis (0.7% agarose in TAE (Tris base, acetic acid and EDTA buffer) and purified by phenol/chloroform extraction. After amplification, cDNA fragments of 200 to 600 bp were size selected using solid phase reversible immobilization magnetic beads.

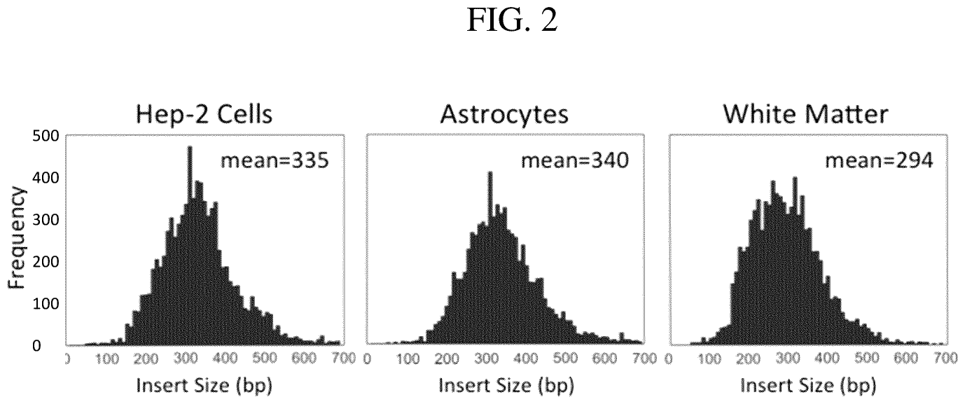

Purified human cDNA amplicons were ligated into linearized pSEX81 or pG8SAET vectors using a molecular cloning method which allows for the joining of multiple DNA fragments in a single, isothermal reaction (Gibson assembly cloning). The Gibson ligation product was then amplified using Phi29 polymerize, digested with Notl-HF and circularized. Circularized ligated phagemids were electroporated into phage display electrocompetent E. coli strain TG1 cells. After electroporation, the cells were suspended in SOC media and cultured for 35 minutes at 37° C. The cells were plated on 15 cm culture plates (1.5% agar, 100 μg/ml carbenicillin, 1% glucose) using glass beads. The plates were incubated at ° C. for 18 hours before the cells were harvested by scraping. Human cDNA inserts contained in the phagemid vectors that were transformed into TG1 bacteria were each independently sequenced to assess the diversity and size of the cDNA inserts. FIG. 2 is a histogram showing the range or distribution of cDNA insert sizes in each of the libraries produced using a cell type or tissue type (e.g., a library produced using mRNA of Hep-2 cells, a library produced using mRNA from astrocytes, and a library produced using mRNA from brain white matter). cDNA insert size is determined during the bioinformatics analysis of deep sequencing results. Each individual cDNA fragment sequenced within individual libraries is identified by their unique nucleotide start and end positions relative to the reference human genome using a custom Python3 script suite designed and developed for this purpose. This combination of genomic coordinates allows the precise identification of unique DNA clones and their sizes. High diversity phagemid libraries (estimated to contain ≥3.6×10⁷ independent cDNA inserts) with 294-340 bp mean insert sizes (FIG. 2) were pooled together using equal numbers of transformed bacteria. These pooled libraries were used for the production of phage particles.

Phage particles were generated using 10¹⁰ bacteria grown in 100 ml of 2YT media supplemented with 1% glucose and 100 μg/ml carbenicillin. Cultures were stopped when their optical densities (0D600) reached 0.4 units. Hyperphage M13 K07ΔpIII helper phage were added at a multiplicity of infection (MOI) of 10:1 to pSEX81 transformed cells, while VCSM13 interference-resistant helper phage were added to pG8SAET transformed cells. The cultures were then incubated for 30 minutes at 37° C. without shaking. The bacteria were pelleted by centrifugation at 2,500×g for 30 minutes and resuspended in 200 mL of fresh 2YT medium supplemented with 100 μg/ml carbenicillin and 10 mM MgCl₂. The superinfected cells were cultured again for 1 hour at 25° C. before kanamycin was added at a final concentration of 70 μg/ml to terminate the proliferation of bacteria not infected with helper phage. After an 18-hour incubation with vigorous shaking, the bacterial cells were removed by centrifugation at 2,500×g for 1 hour. Phage particles were precipitated from the cleared culture supernatant fluid by incubation at 4° C. for 1 hour in the presence of 0.5 M NaCl and 4% PEG8000. After centrifugation, the phage pellet was resuspended in PBS containing 15% glycerol, titrated to quantitate phage numbers and used immediately for immunoprecipitation experiments or stored at −80° C. Using these methods, repeated deep sequencing of the pooled phagemid and phage libraries, and bioinformatics analysis with complexity estimates indicated a library complexity of ≥3.6×10⁷ unique cDNA inserts, with these cDNA inserts representing at least 19,327 identified human genes.

### Example 2

This example illustrates the use of the antigen display library, described in Example 1 above, to identify antigenic epitopes recognized by antibodies in a sample from an individual. In the schematic diagram shown in FIG. 1B, illustrated is contacting the phage display library with a sample containing antibody; immunoselection of phage displayed antigen bound by antibody in the sample, wherein the antibody of the antigen-antibody complex is immobilized on a substrate; and deep sequencing the immunoselected phage for determining the cDNA insert that encodes the antigenic epitope recognized by antibody in the sample.

Aliquots of the pooled phage display library (^{˜}2×10¹⁰ infectious particles) were resuspended in PBS and pre-cleared by adding a suspension Protein A-conjugated paramagnetic beads with rotation at 4° C. for at least 1 hour. After centrifugation to pellet the beads, the phage suspension was harvested, with 1 μL of a biological sample containing or suspected of containing antibody (in this example, human serum or plasma) added to each precleared aliquot of phage before incubation overnight with gentle rocking at 4° C. Aliquots of the Protein A-conjugated paramagnetic beads were suspended in PBS containing 2% ovalbumin (w/v) overnight at 4° C. and washed before being added to the phage/serum mixtures. After 2 hours of incubation at 4° C. with rotation, the beads were pelleted by centrifugation and washed twice with PBS containing 0.1% Tween 20 for 5 minutes to dilute out the unbound phage that were not bound to antibodies. The beads were washed four additional times in PBS containing 0.1% Tween 20 for 10 minutes, then washed twice in PBS containing 0.05% Tween 20 for 15 minutes, with one final wash in PBS containing 0.01% Tween 20 for 10 minutes. The pSEX81 phagemid encodes a trypsin-sensitive protease cleavage site between the cDNA-encoded human protein and the phage protein. Thereby, functional phage particles bound by antibodies were released from the antibody-coated magnetic beads by incubation with 0.5% Trypsin for 15 minutes. pG8SAET phage were released from the antibody-coated magnetic beads by suspending the phage/antibody/bead mixtures in 100 mM glycine (pH 2.5) for 15 minutes.

Non-specific phage binding during the immunoselection step with individual serum/plasma samples was reduced by repeating the phage/antibody selection process a second time to further enhance the specificity of phage selection by antibody. After the phage particles were eluted from the antibody-bound beads, the bound phage from individual samples were amplified by infecting TG1 cells, which were expanded by culturing as previously described herein. After expansion, the TG1 cells were superinfected with the appropriate helper phage to induce phage production. The amplified phage were then selected a second time using the same serum as in their original selection as described above. The phage particles eluted after the second round of selection were used to infect fresh TG1 cells that were then expanded.

Phagemid DNA was extracted from TG1 cells using a commercially available miniprep kit according to the manufacturer's instructions. Because of the way that the human cDNA inserts had to be designed, amplified and manipulated to promote optimized phage diversity, a custom strategy was required for deep sequencing of the cDNA inserts. Custom PCR adapters were designed to PCR amplify the human cDNA inserts within the individual antibody-selected pools of phage DNA. Customized amplicons for pSex81 library sequencing were generated using a custom Index primer comprising SEQ ID NO:13 (5′-CAAGCAGAAGACGGCATACGAGATNNNNNNGTGACTGGAGTTCAGACGTGTGCTCTTCCGATCAA TCCAGCGGCCGCAAC-3′) where NNNNNN indicates a sample-specific DNA barcode for multiplex DNA sequencing (where N is selected from A, T, G, or C at each position), along with a custom Universal primer comprising SEQ ID NO: 14 (5′-AATGATACGGCGACC ACCGAGATCTACACTCTTTCCCTACACGAC GCTCTTCCGATCTCCATGGCCGCCGAGAAC-3′) specific for this application. Customized amplicons for pG8SAET library sequencing included a custom Index primer comprising SEQ ID NO:15 (5′-CAAGCAGAAGACGGCATACGAGATNNNNNNGTGACTGGAGTTCAGACG TGTGCTCTTCCGATCCCGGCGG CCGCAAC-3′) and the same Universal primer as was used for pSEX81 template amplification. PCR was performed using these primers and DNA polymerase under the following conditions for 10 cycles: 94° C. for 20 seconds, 65° C. for 20 seconds, and 72° C. for 25 seconds. PCR amplicons between 200 to 600 bp in size were selected for each sample using solid phase reversible immobilization magnetic beads, quantified, and pooled for nucleic acid sequencing using methods known in the art. Custom designed sequencing primers for this application were a forward primer comprising SEQ ID NO:16 (5′-CCGATCTCCATGGCCGCCGAGAAC-3′) and a reverse primer comprising SEQ ID NO:17 (5′-TCCGATCAATCCAGCGGCCGCAAC) for pSEX81 library sequencing; and a reverse primer comprising SEQ ID NO:18 (5′-CCGATCCCGGCGGCCGCAAC-3′) used for sequencing the pG8SAET library. FIG. 2 is a series of histograms showing the range of cDNA insert sizes from different phage libraries produced based on tissue or cell source (e.g., Hep-2, fetal astrocytes, and brain white matter) of originating mRNA.

For bioinformatics analyses, sequencing reads were first filtered for quality and length using Cutadapt software. Reads with Phred quality scores <20 and lengths <40 base pairs were excluded from the analysis. PCR adapter sequences were then trimmed from the filtered reads using Cutadapt software. Reads were then aligned to the hg19 human genome reference assembly using the Tophat2 aligner and mapper software package. Aligned reads were then annotated, and the number of reads attributed to each gene within each sample library was counted using Htseq-count software. The data analysis script used to filter, trim, align, annotate, and count sequencing reads is available for download online. For data analysis, all sequencing reads that were obtained for each sample library were first grouped into gene (or defined protein domain) bins that were representative of the expressed genes within the original pooled HEp-2, astrocyte and brain display library used for phage immunoprecipitations. Some bins contained relatively high numbers of reads, some bins were empty, while other bins reflected a spectrum of read numbers. It was thereby possible to quantify the number of sequence reads within each bin of each sample library after phage immunoprecipitations relative to the number of sequence reads within each bin in the original pooled library. There was no obvious or statistical correlation between the number of reads within bins of the antibody selected libraries relative to the original pooled library, demonstrating that the selection process selectively enriched for subsets of specific gene (or defined domain) sequences. Moreover, it was possible to quantitate the relative number of reads obtained within each bin and use that number as a quantitative measure of the intensity of antibody selection that was obtained with that biological sample.

The total number of reads obtained for each gene (or defined protein domain) bin across all sample libraries was then normalized to account for the inherent variability in sequencing depths obtained across different libraries and sequencing runs. The number of reads obtained for each gene (or defined domain) domain were determined as above. The bins were then rank-ordered, with the bin having the highest number of reads at the top (representing the 100^{th} percentile) and the bin having the lowest number of reads at the bottom (representing the 1^{st} percentile). The number of reads obtained in the bin at the 85^{th} percentile was then determined. The 85^{th} percentile value was empirically determined to fit the sequencing data better than using total, mean, or median (50^{th} percentile) sequencing read numbers due to the distribution in read numbers across all sequenced samples. The number of reads obtained for each gene (or defined protein domain) bin in a given sample were then divided by the number of reads at the 85^{th} percentile for that sample. This method of normalization means that for each sample, the genes among the top 15% most highly expressed genes (or defined protein domain) bins in the sample library have normalized values >1, and the gene (or defined protein domain) bins among the bottom 85% of all expressed genes (or defined protein domain) bins have normalized values <1. Normalizing sequencing counts between samples therefore permits the direct comparison of read numbers for each gene (or defined protein domain) bin among all samples. The normalized number of reads for each gene (or domain), as determined above, was then converted into pseudocounts ≥0 to more accurately reflect the raw number of sequencing reads obtained for each gene (or domain), across every sample. Once the number of reads at the 85^{th} percentile was determined for each sample, the geometric mean for sequencing reads at the 85^{th} percentile among all samples was determined. Pseudocounts were then obtained by multiplying the normalized number of reads for every gene (or domain) by the geometric mean number of sequencing reads at the 85^{th} percentile among all samples. Using this method across all samples, the number of sequencing reads at the 85^{th} percentile in each sample is then equivalent to the calculated geometric mean value for all samples. Finally, pseudocounts were log-transformed using log-base 10 for further analysis.

The edgeR software package was used to identify genes having significantly increased counts among disease cohorts. After count normalization, the total number of gene (or defined protein domain) bins was reduced by removing bins with low counts across all samples. Low counts were determined as bins having less than 15 counts per 10⁶ total normalized reads for that individual serum sample. Bins within each serum sample were also removed from the analysis if the bin counts were less than 2 fold higher (by edgeR software) than the counts obtained for a panel of background/control samples. Background/control samples were processed along with the serum samples in each assay to identify proteins/domains that were non-specifically enriched or bound in the absence of added human serum. After the removal of low count bins from the protein/domain list, sample-wise common dispersion and protein/domain-wise dispersion was quantified for each bin. A statistical exact test adapted for negative binomial distributions (edgeR) was then used to calculate fold change differences for the background values versus each serum sample bin and to assign corresponding p-values for each bin. All bins having mean counts across each serum cohort that were <2 fold higher than the mean counts of the background controls were then removed from the analysis. This cycle was repeated to identify disease cohort protein/domain bins that were significantly different from the healthy control cohort. At the end, bins with mean counts 2-fold higher in disease samples as compared to healthy samples and with false discovery adjusted p-values >0.05 were selected as disease-specific. This subset of protein/domain bins was used to generate disease-associated autoantibody signatures for patients and subsets of patients.

### Example 3

### Antibody Signatures

This example illustrates the use of the antigen display library to identify antigenic epitopes recognized by antibodies in a sample from an individual (as described in Examples 1 & 2 herein) to generate antibody signatures. Thus, in addition to determining gene products identified by antibodies contained within a sample, the data generated using the current bioinformatics pipeline can also be used for mapping and predicting antibody-binding sites within specific regions, domains, and epitopes (conformational or linear) of the target proteins. This can be achieved over a broad spectrum of resolution down to the amino acid sequence level by using additional analysis procedures. For this purpose, each individual DNA fragment sequenced within the individual libraries was identified by their unique nucleotide start and end positions relative to the reference human genome using a custom Python3 script suite designed and developed for this purpose. This combination of genomic coordinates allows the precise identification of unique DNA clones for mapping and predicting antibody binding sites at high resolution. As one example, individual unique cDNA sequences can be binned together if their nucleotide start or end positions differ by <100 bases. In the current sequencing example, this approach permitted the binning of antibody-isolated protein fragments (generated by clustered cDNAs) from the pooled human cDNA-containing phage libraries into ^{˜}5×10⁶ individual overlapping protein domain bins for analysis. The numbers of antibody-selected cDNA fragments falling within each bin and overlapping domain bins can be quantified by bioinformatics analysis so as to generate maps showing the most likely antibody binding regions and epitopes within each target protein domain.

Delineating each gene product (or protein domain) recognized by antibodies in a biological sample from an individual, while also quantifying the frequency at which each protein product is identified by antibodies within each sample, generates an antibody signature for each individual. Because all of the phage clones selected by each antibody sample are derived from the same original pool of human cDNA-containing phage libraries, direct comparisons are allowed between each serum-specific phage pool after phage immunoprecipitations. Because the phage libraries containing cDNA derived from each of the individual cell type or tissue type (e.g., H Ep-2, astrocyte, and brain) were also individually sequenced whereby individual cDNA clones from each library are identified, the cell source of each individual phage clone and its protein domain product can be determined as unique to one cell source or shared by two or more cell types. Thereby, different antibody signatures between individuals can be quantitatively compared directly at the gene or protein level or at even higher resolution.

### Example 4

In this Example, illustrated is the use of the compositions and methods described in Examples 1-3 herein to generate antibody signatures from antibodies contained in samples from individuals with various autoimmune diseases, and as compared to antibody signatures from healthy individuals. Biological samples were from human donors after appropriate informed consent and protocol approval was obtained.

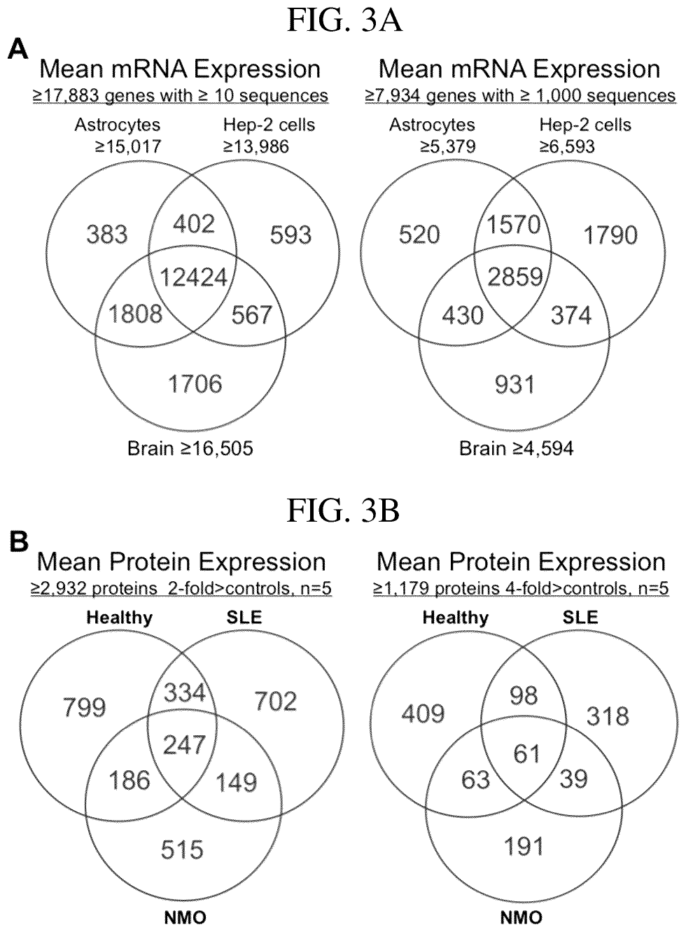

Immunoselections using the phage display libraries, as described in Examples 1-3, were performed using samples obtained from individuals with autoimmune disease diagnosed as Neuromyelitis optica (NMO), using samples obtained from individuals with autoimmune disease diagnosed as lupus (SLE), and using samples from healthy individuals with no overt symptoms of any autoimmune disease. Analyzed was gene expression based on mRNAs isolated from the original source material (human astrocytes, brain white matter, and Hep-2 cells) prior to phage display library production. A Venn diagram (FIG. 3A) shows the analysis of such genes differentially expressed by the cells from each original source of mRNA with 10 sequenced reads or 1,000 sequenced reads per gene transcript. The Venn diagram shows the number of genes expressed solely by an original source material versus the number of shared genes expressed between multiple original source materials. For comparison, FIG. 3B is a Venn diagram showing the analysis of proteins encoded by genes differentially expressed by the cells of the original source of mRNA (Hep-2, fetal astrocytes, and brain white matter) after phage display library production, pooling of phage display libraries produced, and immunoselection with serum from either healthy individuals, serum from individuals with systemic lupus erythematosus, or serum from individuals with Neuromyelitis optica. The proteins identified in FIG. 3B represent the number of gene products immunoselected by serum samples that were enriched (mean that is ≥2-fold or ≥4-fold higher) among each individual cohort (healthy, SLE, or NMO) relative to the mean counts observed among negative control samples where CD20 monoclonal antibody or no antibody was used in the phage selection assays. The Venn diagram represents the relative segregation of all enriched immunoselected genes among the serum samples from the different cohorts.

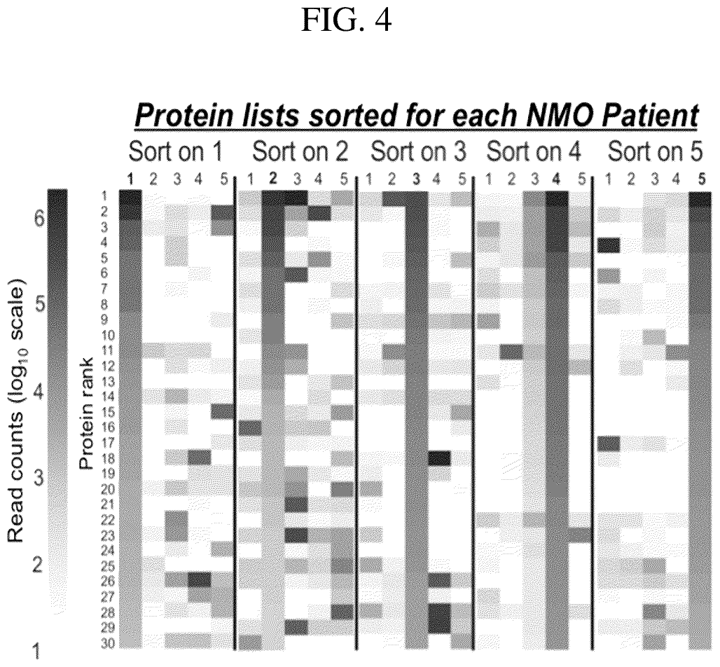

Immunoselections and bioinformatics analyses were used to generate antibody signatures for 5 individuals diagnosed with Neuromyelitis optica relative to negative control samples where CD20 monoclonal antibody or no antibody was used in the phage selection assays. Bioinformatics was used to sort the genes identified through immunoselection and from high counts to low counts, in this cohort of individuals. The top 30 proteins encoded by genes selected most frequently by antibodies contained in each individual sample were compared with the counts observed for the same proteins/genes selected by antibodies contained in serum samples from the other individuals in this cohort. Shown in FIG. 4 is a heatmap illustrating the antibody signatures generated for the 5 individuals diagnosed with NMO, where intensity of color reflects the relative number of counts for each protein observed from immunoselection and analysis of each sample expressed on a logarithmic scale. Thus, in comparing antibody signatures of individuals diagnosed with the same disease (a cohort), detected are antibodies from each individual of the cohort that recognize the same antigenic domain or epitope (potentially, an autoantigen) as well as antigens that are differentially expressed and recognized by antibodies from an individual as compared to that of other individuals in the cohort. These antibody signatures, or the individual gene products identified by the antibody signatures, may have potential use as biomarkers, or prognostic, diagnostic or therapeutic uses, for NMO.

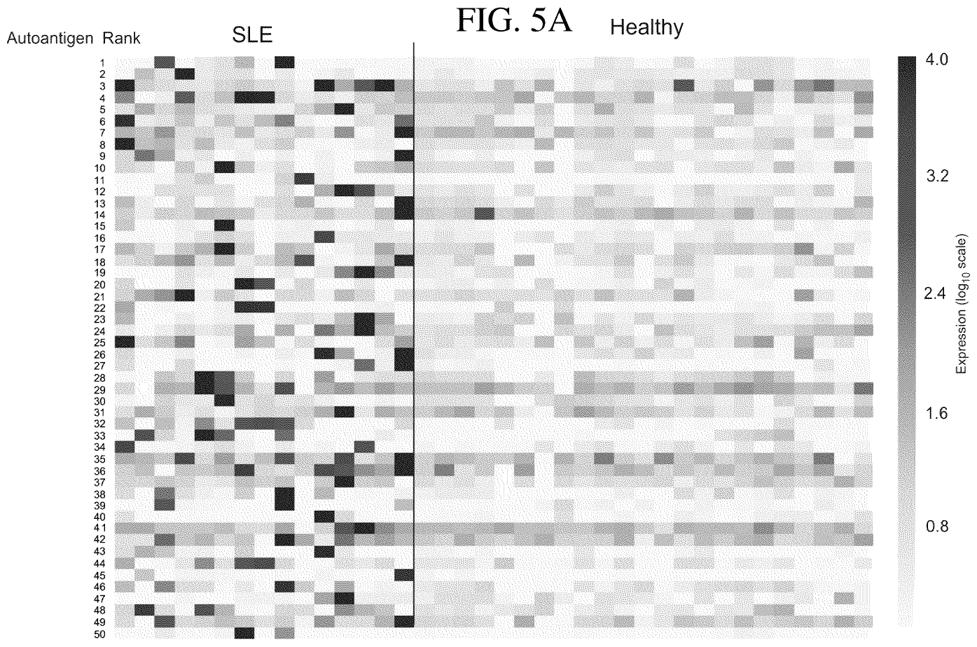

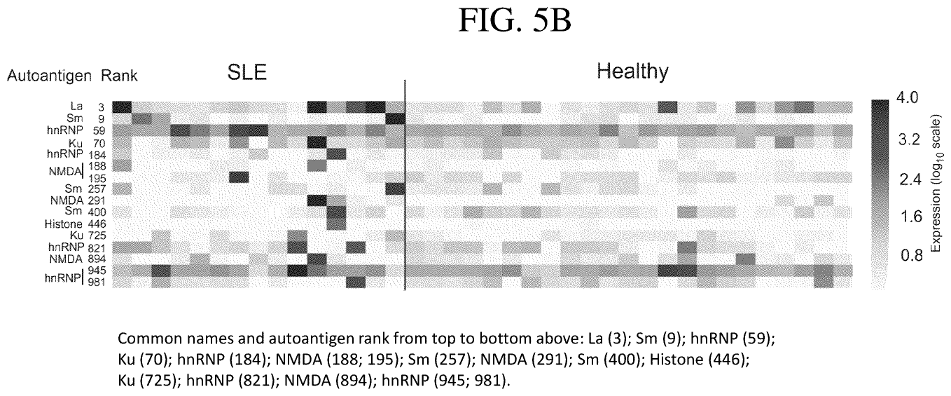

Immunoselections and bioinformatics analyses were used to generate antibody signatures for individuals diagnosed with SLE (“SLE cohort”), as well as antibody signatures for 23 healthy individuals (“Healthy cohort”) for comparison purposes. Bioinformatics was used to sort the gene products identified through immunoselection, with the number of immunoselected phages representing each gene counted for each sample tested. The top 50 genes selected most frequently by antibodies contained in each individual sample of the SLE cohort were compared with the counts observed for the same genes selected by antibodies contained in serum samples from the other individuals in the SLE cohort. The same list of “SLE” protein ranking from high to low was used for comparing the same genes selected by antibodies contained in serum samples from the healthy individuals. Shown in FIGS. 5A-5B are heatmaps illustrating the antibody signatures generated for the 15 individuals diagnosed with SLE, as compared to the antibody signatures generated for the healthy individuals, where intensity of color reflects the relative number of counts for each gene observed from immunoselection and analysis of each sample expressed at a logarithmic scale. Thus, in comparing antibody signatures of individuals diagnosed with the same disease (e.g., the SLE cohort), detected are antibodies from each individual of that cohort that recognize the same antigenic epitope (potentially, an autoantigen) as well as antigens that are differentially expressed and recognized by antibodies from an individual as compared to that of other individuals in the same cohort. In that regard, FIG. 5B is a heatmap illustrating antibody signatures for the individuals in the SLE cohort, compared to antibody signatures for the individuals in the Healthy cohort, as shown in FIG. 5A, except that selected are autoantigens known to be associated with SLE. Thus, antibody signatures, or the individual gene products identified by the antibody signatures, have potential use as biomarkers, or prognostic, diagnostic or therapeutic uses, for SLE.

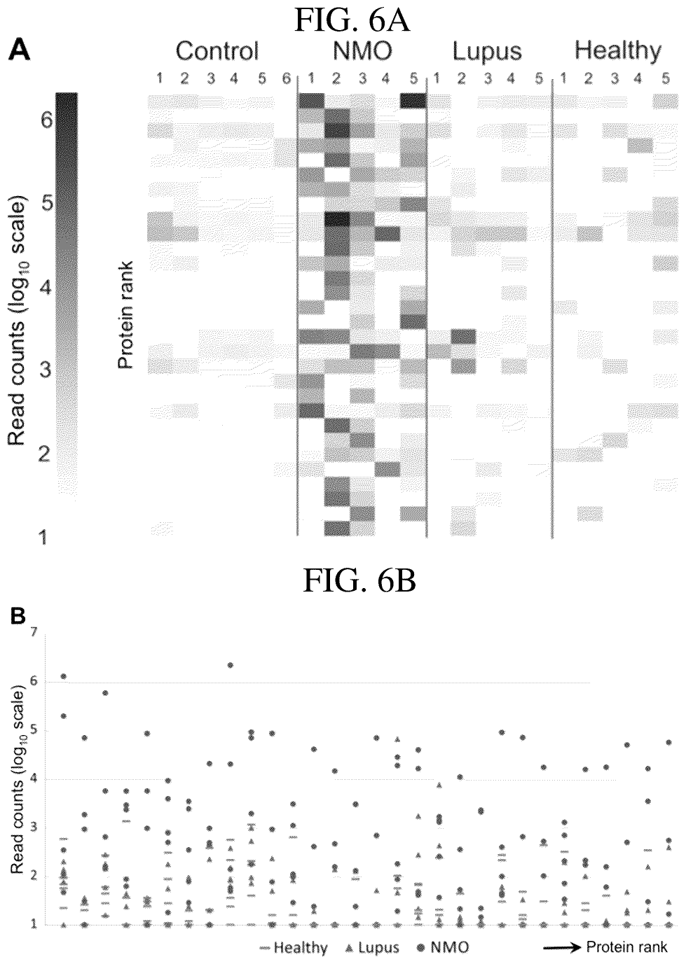

The antibody signatures may also be compared between different disease cohorts. For example, FIG. 6A is a heatmap illustrating antibody signatures for 5 individuals with NMO, 5 individuals with SLE, and 5 healthy individuals relative to 6 negative control samples where CD20 monoclonal antibody (n=3) or no antibody (n=3) was used in the phage selection assays. Shown are 30 gene products selected most robustly by antibodies contained in samples from individuals with NMO. Intensity of color reflects the relative number of counts for each gene observed for each sample expressed at a logarithmic scale. While it is clear that the antibody signatures are distinct for each disease cohort, and as compared to the Healthy cohort and controls, noted are some antibodies from each disease cohort (NMO cohort and SLE cohort) that recognize the same gene product, although at different frequencies of detection/expression. The relative differences between individuals and cohorts are also quantitative, with differences between individuals ranging from over a 100,000- to 1,000,000-fold, to equivalence (FIG. 6B).

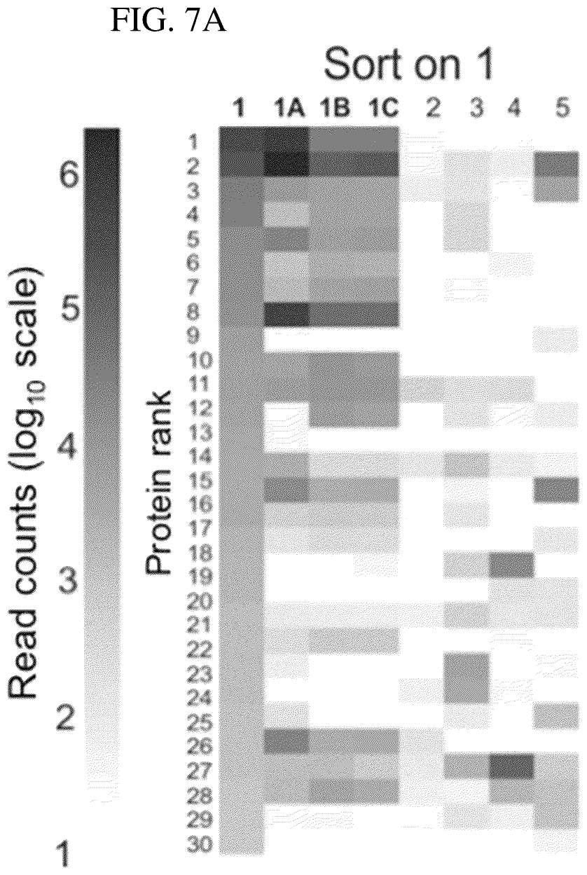

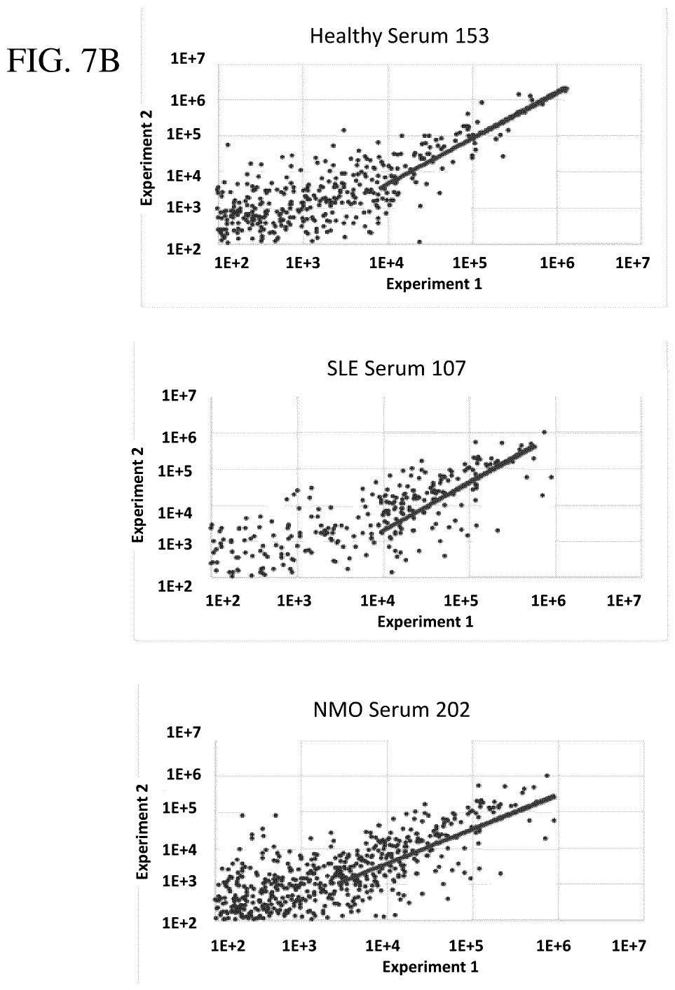

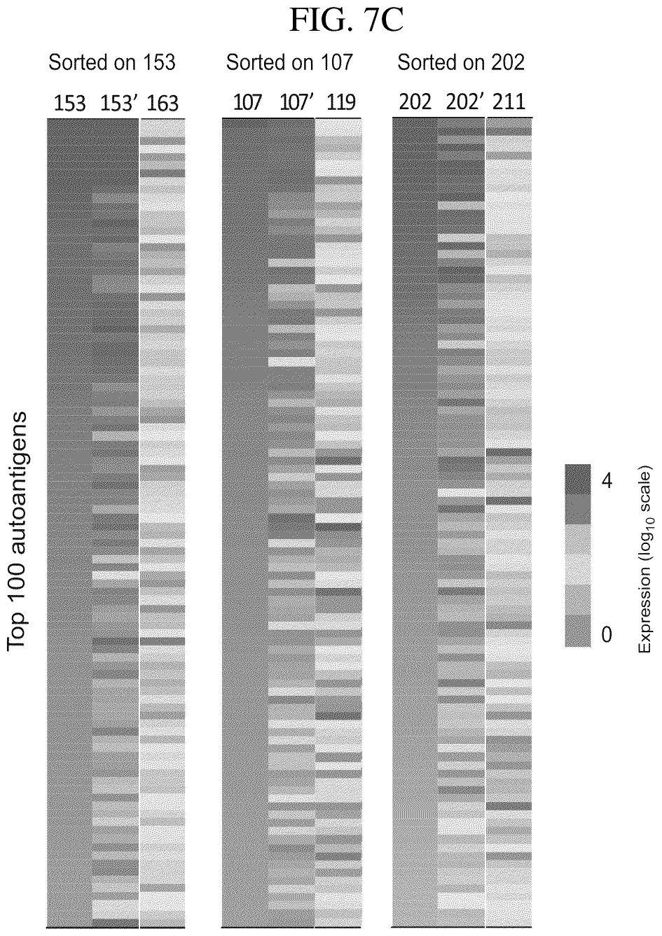

The reproducibility of generating antibody signatures was first analyzed as illustrated in FIG. 7A using antibodies from the same sample of individual 1 with NMO, but from 4 independent immunoselection assays (“1”, “1A”, “1B”, and “1C”). Antibody signatures generated using antibodies from individual “1” and four other individuals with NMO (“2”, “3”, “4”, “5”) were sequenced at high depth, while the sequencing runs for 1A, 1B, and 1C were at 20-fold lower depth. Shown are 30 gene products selected most robustly by antibodies from individual “1” with NMO. Intensity of color reflects the relative number of counts for each gene product observed for each sample expressed at a logarithmic scale. Even though the samples from individual “1” with NMO were from different assays and were sequenced at different depths, the gene signatures and gene products isolated in each assay were similar and were distinct from those obtained from the four other individuals with NMO (“2”, “3”, “4”, “5”). This experiment shows that antibody signature production is very reproducible between immunoselection assays.

Illustrated in FIG. 7B is a comparison of autoantigen counts obtained using the same three serum samples obtained from a healthy control and individuals with SLE or NMO for immunoselection in two independent experiments. The three panels demonstrate how well counts from one experiment mirror the relative counts obtaining during a subsequent experiment. In all three comparisons, proteins with high counts showed minimal variation between the two different assays and exhibited the correlation trend indicated by the diagonal line as calculated using least squares methods. However, proteins with lower counts were more variable due to up to four-fold differences in the diversity of the sequenced reads, batch-to-batch effects, and the lower sequencing depths with these samples. Nonetheless, heat map comparisons for the 100 most abundant autoantigen specificities in experiment 1 for sera 153, 107 and 202 were remarkably similar (FIG. 7C). The heat map generated using serum from a different individual within each cohort shown that the autoantibody profiles of samples 163, 119, 211 are distinct. Moreover, these results reinforce the observation that each individual possess a unique autoantibody ‘signature’. Nonetheless, antibody signatures were reproducible between immunoselection assays, thereby allowing comparisons between individual samples and independent assays.

### Example 5

In this Example, illustrated is the use of the compositions and methods described in Examples 1-3 herein to identify target proteins and their domains or epitopes reactive with antibody samples of known or unknown specificity. An antibody sample with defined specificity to an antigen known to exist in the phage display library was used to select the known antigen using the described selection and bioinformatics analysis. To this end 300 ng of each of 15 rabbit polyclonal antibody samples with specificities to 15 human proteins (AB12, CALD1, UBA1, NONO, PCNA, ATN1, CAV1, DDX5 ITGB1 LDHB MAPK9, RAC1, SHC1, SOS1, THRAP3) displayed in the library were mixed with 2.4 mg of a chimeric human antibody against a protein not present in the library. This antibody cocktail was used for phage selection. The antigens identified by the rabbit antibodies were displayed at low to medium frequencies in the parental phage display library, ranging between 10 to 1,000 phage clones per protein in each immunoprecipitation reaction. For comparison, common cytoskeleton proteins of the actin family were represented by 7,000 to 12,000 clones. The commercial rabbit antibodies were elicited using 50 amino acid peptides originating from the C-terminal regions of the proteins. Rabbit antibodies were used because of their similarity in binding to protein A conjugated paramagnetic beads with human IgG antibodies.

Antigen selections were performed as described in Example 2. The phage/antibody selection process with phage amplification in TG1 cells repeated three times to investigate the extent of phage/antigen enrichment after each selection step. After each expansion, a fraction of the TG1 cells was reserved for phagemid purification and subsequent sequencing, while the rest were superinfected with the appropriate helper phage to induce phage production. The amplified phage were then reselected. cDNA inserts within phagemids extracted from the TG1 cells were identified through MiSeq Illumina sequencing and bioinformatics analysis. Sequencing reads were aligned to the reference human genome, counted and normalized. The enrichment was compared against clone counts of the selected phage in the starting library with no selection.

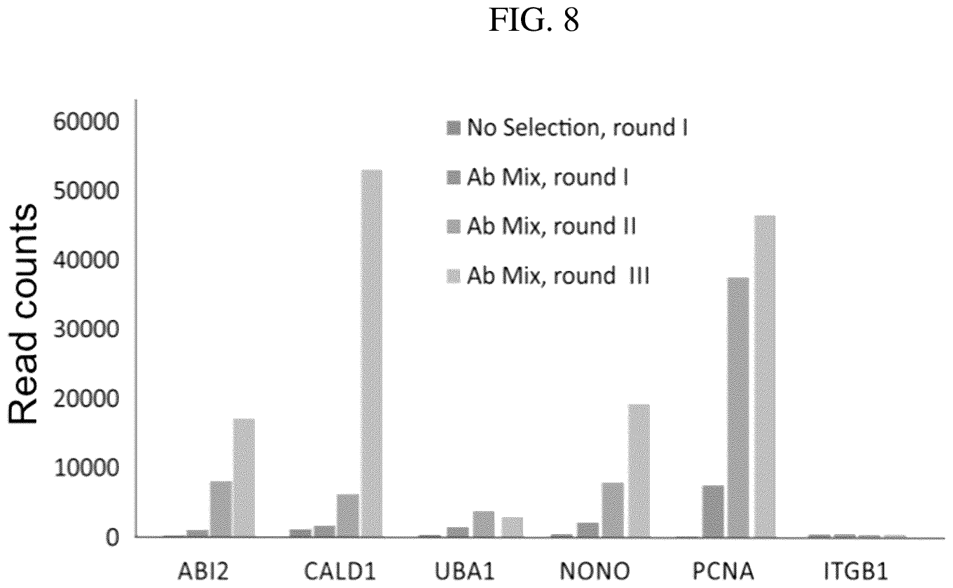

Sequencing data analysis demonstrated that AB12, CALD1, UBA1, NONO, PCNA were highly enriched after two rounds of selection with enrichment 26-, 5.3-, 9.3-, 15.8-, and 250-fold, respectively (FIG. 8). Phage encoding ATN1, DDX5, and MAPK9 sequences were enriched at lower levels of 1.9-, 1.8-, and 3.3-fold, respectively (data not shown). By contrast, phage expressing ITGB1, LDHB, RAC1, SHC1, SOS1, and THRAP3 protein domains were not selected by rabbit antibodies. These negative results were likely due to the low representation of the appropriate phage clones encoding the polypeptides used as immunogens as SHC1, RAC1 proteins were well represented within the libraries. Alternatively, the anti-peptide antibodies may bind linear epitopes that are not appropriately displayed by the domain-sized proteins expressed by phage. While a third selection step improved the detection signal, the overexpansion of some clones demonstrated that two rounds of selection would be sufficient to detect the expansion of phage expressing diverse proteins without reducing the diversity of immunoselected phage clones during amplification. Therefore, the method is suitable for the identification and characterization of antibody specificities within complex antigen mixtures.

### Example 6

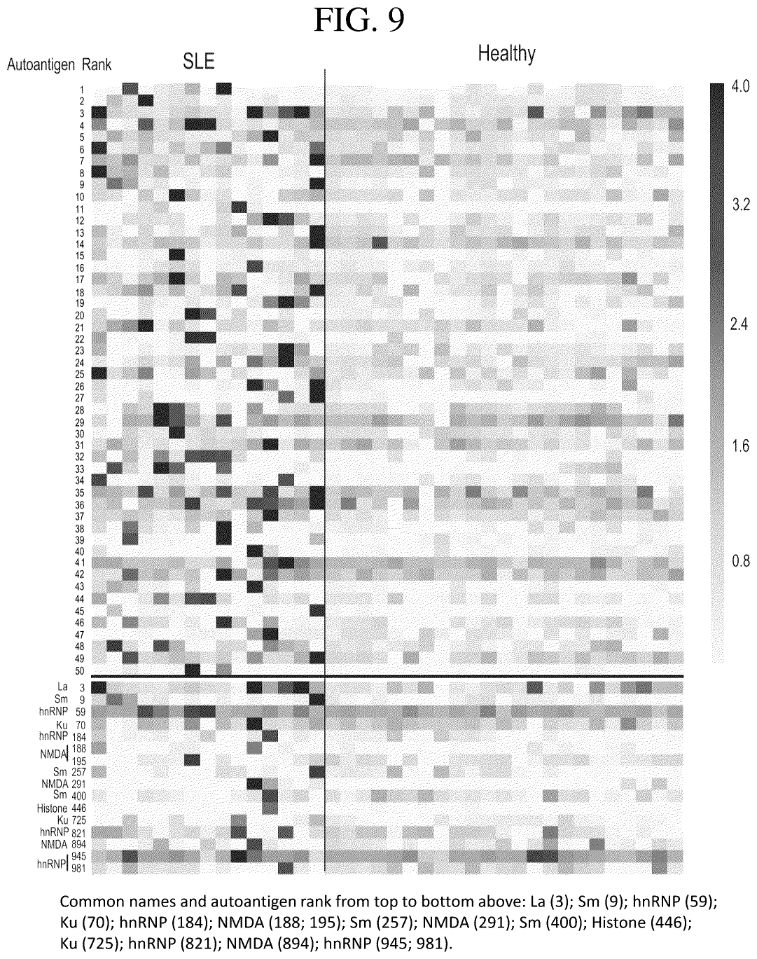

Immunoselections and bioinformatics analyses were used to generate antibody signatures for 15 individuals diagnosed with SLE (“SLE cohort”), as well as antibody signatures for 23 healthy individuals (“Healthy cohort”) for comparison purposes. Bioinformatics was used to sort the gene products identified through immunoselection, with the number of immunoselected phages representing each gene counted for each sample tested. The top 50 genes selected most frequently by antibodies contained in each individual sample of the SLE cohort were compared with the counts observed for the same genes selected by antibodies contained in serum samples from the other individuals in the SLE cohort. The same list of “SLE” protein ranking from high to low was used for comparing the same genes selected by antibodies contained in serum samples from the healthy individuals. Shown in FIG. 9 is a heatmap illustrating the antibody signatures generated for the 15 individuals diagnosed with SLE, as compared to the antibody signatures generated for the healthy individuals, where intensity of color reflects the relative number of counts for each gene observed from immunoselection and analysis of each sample expressed at a logarithmic scale. Thus, in comparing antibody signatures of individuals diagnosed with the same disease (e.g., the SLE cohort), detected are antibodies from each individual of that cohort that recognize the same antigenic epitope (potentially, an autoantigen) as well as antigens that are differentially expressed and recognized by antibodies from an individual as compared to that of other individuals in the same cohort. In that regard, the lower panel heatmap illustrates antibody signatures for the individuals in the SLE cohort, compared to antibody signatures for the individuals in the Healthy cohort except that the listed gene-products are autoantigens known to be associated with SLE. Also shown are the relative ranks of these autoantigens among all autoantigens selected from the pooled antigen display library. Two known autoantigens, La and Sm, were among the top ranked 50 autoantigens. The remaining 14 autoantigens shown in the lower panels ranked between 59 to 981 among the selected autoantigens. Thus, the majority of known proteins selected in the current antigen display system are not known autoantigens. Thereby, the antibody signatures, or the individual gene products identified by the antibody signatures, have potential use as biomarkers, or prognostic, diagnostic or therapeutic uses, for SLE.

### Example 7

In this Example, illustrated is the use of the compositions and methods described in Examples 1-5 herein to determine target autoantigens recognized by 6 reference standard sera obtained from the US Centers for Disease Control (IUIS ANA standards; http://asc.dental.ufl.edu/ReferenceSera.html) that represent the majority of recognized Anti-Nuclear Antibody (ANA) staining patterns in immunofluorescence assays of HEp-2 cells (www.ANApatterns.org). In this example, autoantibody signatures were validated using standard sera that include antibodies with specificities for known target molecules as previously identified by other labs. Antigen phage libraries were prepared as in Example 1, with antigen selections performed as in Examples 2 and 3. Serum aliquots were incubated with the antigen library and antigen/antibody complexes were selected using protein A-conjugated paramagnetic beads. The selection process with phage amplification was repeated two times. After each expansion, the TG1 cells were superinfected with Hyperphage helper phage to induce phage production. The amplified phage were then reselected using an additional serum aliquot. cDNA inserts within phagemids were identified through NextSeq Illumina sequencing and bioinformatics analysis as described in Example 2. Sequencing reads were aligned to the reference human genome, counted and normalized. Enrichment of protein counts was compared between ANA serum samples and background control samples that had no serum antibody included and serve to identify proteins that bind non-specifically to the antibodies, protein A beads or other system components. The proteins with significant enrichment over background controls were identified as ANA positive autoantigens and were used for further analysis.

The reference sera used for this analysis are known to react with: the SSB/La autoantigen; U1-ribonucleoprotein (RNP) recognized as one or several autoantigens including SNRNP70, SF362, SNRPA, SNRPB, SNRPC; the PM/SCL sera recognizes one or several autoantigens including EXOSC10, EXOSC9, EXOSC8, EXOSC7, EXOSC6, EXOSC5, EXOSC4, EXOSC3, EXOSC2, and EXOSC1; antinuclear autoantigens (ANA) reactive sera identify one or more SSB, SSA, and TROVE2 autoantigens; serum reactive with Sm recognize one or several autoantigens including SNRPB, SNRPD1, SNRPD2, and can cross-react to RNP recognizing one or many SNRNP70, SF362, SNRPA, SNRPB, SNRPC autoantigens; centromere-specific sera may react to one or multiple CENPA, CENTPB, and CENTPC autoantigens.

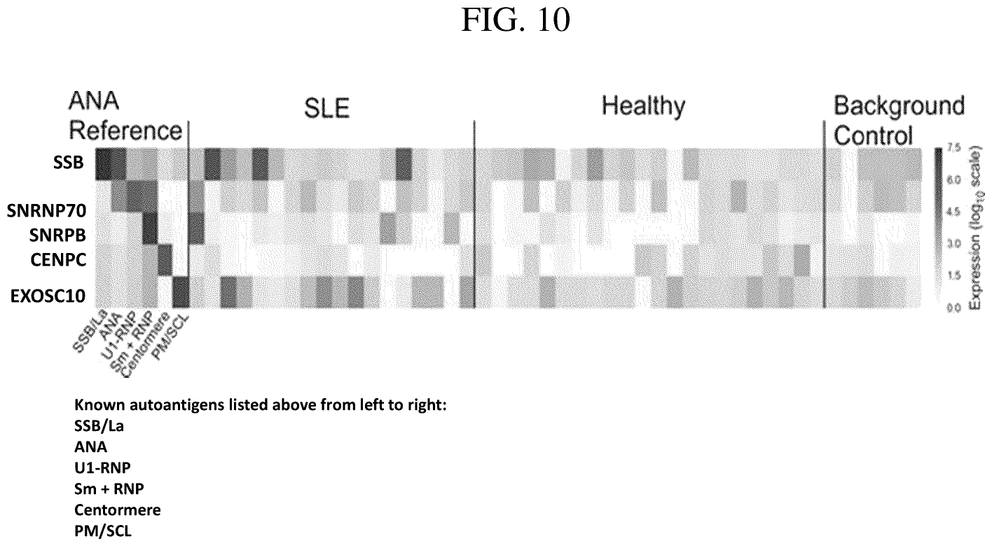

Assay bioinformatics analysis demonstrated that the SSB protein was identified by antibodies contained in two ANA reference serum samples, one reactive with the SSB/La autoantigen and another reactive with ANA (FIG. 10). Notably, antinuclear autoantigens include the SSB protein that binds to singe stranded DNA along with other proteins. The SSB autoantigen was also identified in three sera derived from SLE patients but not in the sera from healthy individuals or background samples. Similarly, antibodies from U1-RNP reactive sera selected the SNRNP70 autoantigen, which is a component of the spliceosomal U1 snRNP. One serum sample in the SLE cohort had elevated counts for this autoantigen as well. Antigen profiling of the centromere-reactive serum using the current antigen selection assay identified antibodies that specifically recognize the CENPC centromere component as a target. Similarly, EXOSC10 was identified to be a molecular target of the exosome-reactive reference serum.

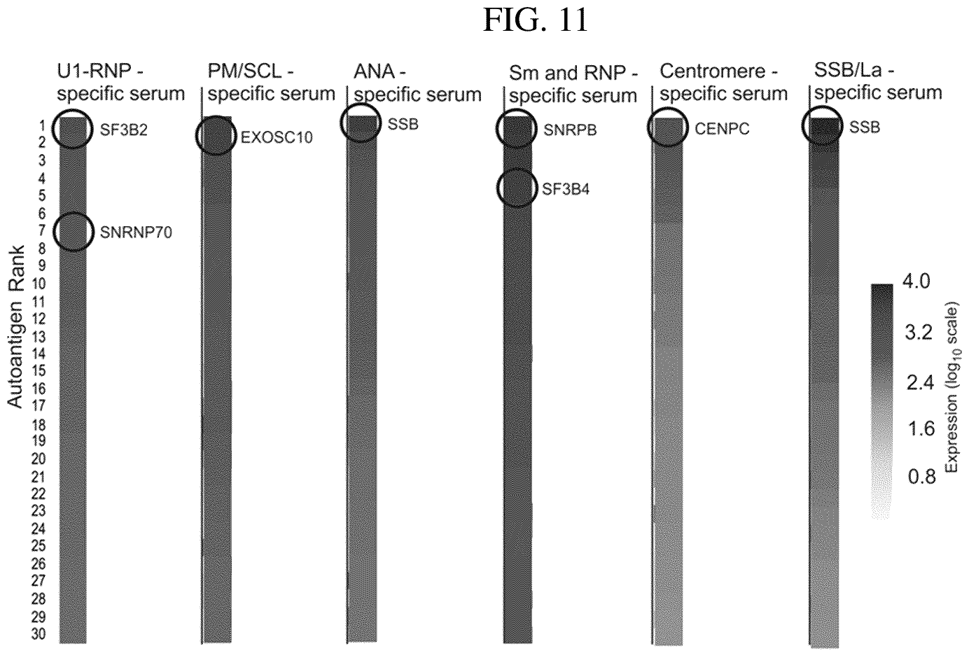

FIG. 11 demonstrates that antibodies contained within the reference sera predominantly select the target autoantigens responsible for the specificities. This selection results in significant enrichment of corresponding gene product in comparison to the other genes within the same sample. Thus the genes encoding the targets are ranked on the top when autoantigens selected by in individual sera sorted from the most enriched to the least. For example, SSB, CENPC, SNRPB, and SF3B2 autoantigens all have the highest counts in the respective sera. ECOSC10 is ranked second. Noteworthy, the reference serum samples demonstrated multiple other autoantibody targets in addition to the previously described specificities. Therefore, the method is suitable for the identification and characterization of antibody specificities within patient serum samples.

### Example 8

This example illustrates the ability of the antigen display system to identify and quantify autoantibody specificities at levels below those identified by conventional Enzyme-Linked Immunosorbent Assays (ELISA), a standard immunological assay technique making use of an enzyme bonded to a particular antibody or antigen. The SSB/La autoantigen is a 47 kDa product of the SSB gene with clinical significance as a marker of multiple autoimmune conditions including SLE and Sjogren's syndrome. This RNA-bind protein contains a helix-turn-helix (HTH) La-type RNA-binding domain at amino acid positions 7-99, flanked by a RNA-Recognition Motif (RRM1) domain at positions 111-187 that is followed by a second RRM2 domain as validated by the SSB crystal structure. Diagnostic ELISAs to measure SSB/La-specific serum autoantibodies are readily available so this autoantigen was used to further validate the current antigen display system. High antigen display assay counts for SSB/La were found in three sera from the SLE cohort an in two ANA reference serum samples, with a broad spectrum of SSB/La-specific autoantibody levels identified in select sera from healthy and patient cohorts as described in Examples 4 and 6.

Thirty serum samples were selected to represent the spectrum of SSB reactivities that were quantified using the current antigen display system. These sera were also evaluated using commercial diagnostic ELISA tests for serum anti-SSB/La autoantibodies. The ELISA plate was coated with full-length SSB/La protein, blocked to prevent non-specific antibody binding, and was subsequently incubated with diluted serum samples as directed by the manufacturer. The amount of SSB-specific autoantibody bound to the plate was measured using a secondary anti-human IgG antibody preparation conjugated with either horseradish peroxidase or alkaline phosphatase. Standardization controls and guidelines for differentiation of the SSB/La positive and negative serum samples were provided by the manufacturer; sera with ELISA values >30 U/mL were considered positive. Similar, if not identical, results were obtained for each serum sample in both ELISA tests based on measured concentrations of anti-SSB antibodies.

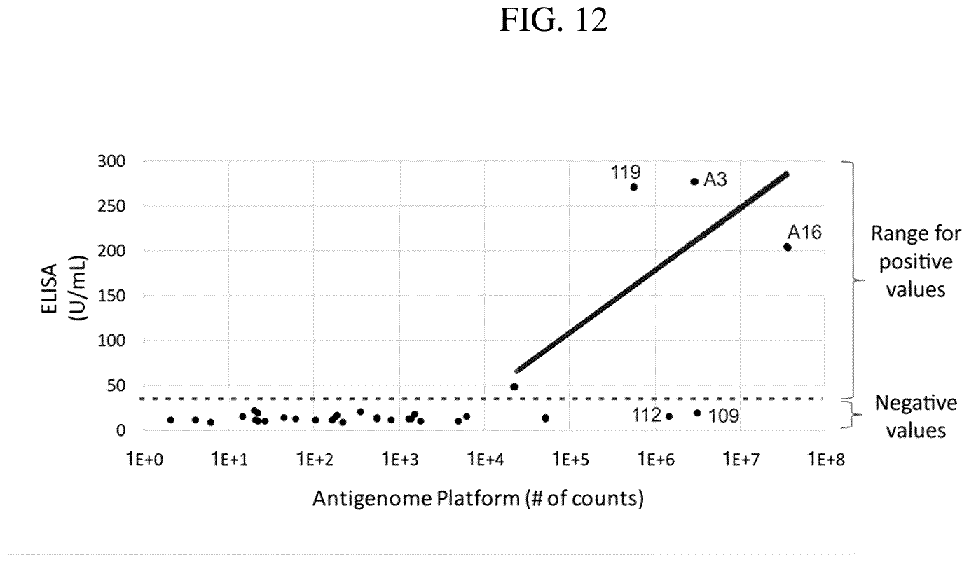

Four of the thirty serum samples tested ELISA positive for SSB/La reactivity (FIG. 12). As examples, serum sample 119 from a patient with SLE, and ANA standard sera A3 and A16 were SSB strongly positive by ELISA and generated high counts in the current antigen display system, while serum samples 109 and 112 from patients with SLE were negative by ELISA, but generated high counts in the current antigen display system. One patient's serum had low positive reactivity with SSB/La in the ELISA assay, but >10000 counts in the current antigen display system, while another patient's serum was negative in the ELISA, but generated even higher counts in the current antigen display system. Thereby, the current antigen display and selection assay was able to identify all of the serum samples that were identified as positive by ELISA for SSB/La autoantibodies. The best fitting line representing these four positive sera was determined using the linear least squares fitting technique, which indicates that the sensitivity for detecting SSB/La autoantibodies in the ELISA is several orders of magnitude lower than the sensitivity of the current antigen display system. Consequently, the current antigen display and selection system has the capacity to identify more serum samples as SSB/La positive than to the diagnostic ELISA.

### Example 9

This example further illustrates the ability of the antigen display and selection system to identify and quantify autoantibody specificities at levels below those identified by conventional diagnostic ELISA tests, and also illustrates the ability of the antigen display system to identify and map antibody binding epitopes of target antigens. Because of the failure of the clinical ELISA to identify anti-SSB/La antibodies in patient samples 109 and 112 (FIG. 12), the pooled human antigen display library utilized for serum sample screening as described in Example 1 was analyzed for the expression of protein domains representing the SSB/La autoantigen.

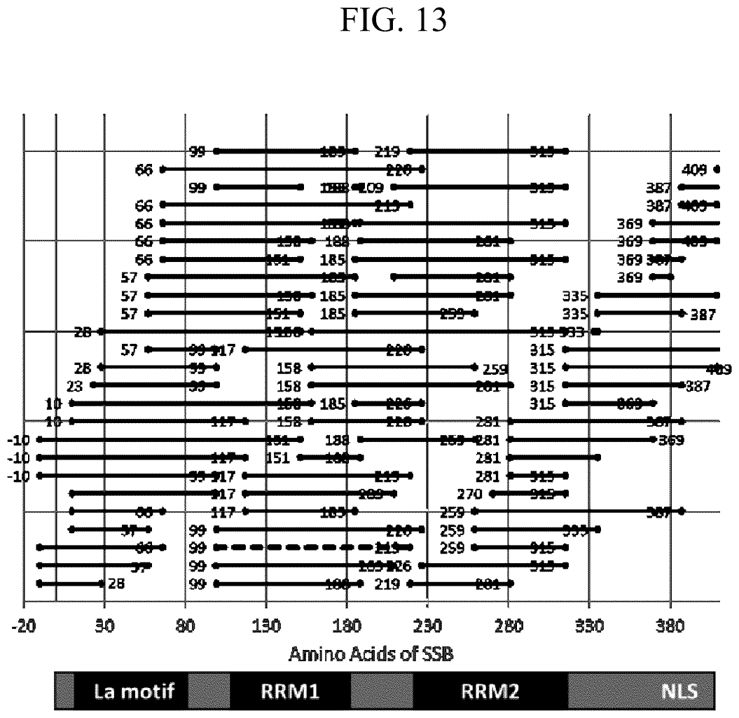

A compendium of the unique SSB/La protein domains identified within the pooled antigen expression library is illustrated in FIG. 13. Individual unique cDNA insert sequences (protein domains) were binned together if their nucleotide start or end positions differed by <100 base pairs. In this example, this approach permitted the binning of protein fragments (generated by clustering cDNAs from the pooled human cDNA-containing phage libraries) into ^{˜}70 individual overlapping protein domain bins. The protein domain bins are demarked by the average first and last encoded amino acid positions of the fragments. These results demonstrate the complexity of the protein domains represented within the pooled antigen display libraries, as well as the structural diversity of the fragments available for selection in each assay by antibodies present within an individual patient's serum. Importantly, a large number of fragments cover the entire La-type RNA binding and two RRM domains and should thereby enable the formation of conformational antibody-binding epitopes within these three different structural units. Moreover, the expression of independent protein domains enables the binding of antibodies that may not bind the intact full-length protein due to conformational constraints and the localization of flanking domains as demonstrated by the crystal structure of the SSB/La protein. Patients may also generated autoantibodies with reactivities against SSB/La sequences and domains that are exposed during protein degradation at sites of cell and tissue destruction.

The dominant SSB domain fragment selected by serum autoantibodies from the antigen expression libraries is indicated as a dashed line in FIG. 13. In fact, eleven of the twelve serum samples with the highest reactivities against SSB/La in the current antigen selection assay including the two sera (109 and 112) with high counts that were ELISA negative (FIG. 12) were reactive with the protein fragment encasing the RRM1 domain (fragment 99-219). The amino terminal HTH La-type RNA-binding domain and the RRM1 domain partner to form the RNA binding region of SSB (fragment 99-219). Thus, the RRM-1 domain is a major substrate for autoantibodies with the sera tested in the current antigen display system.

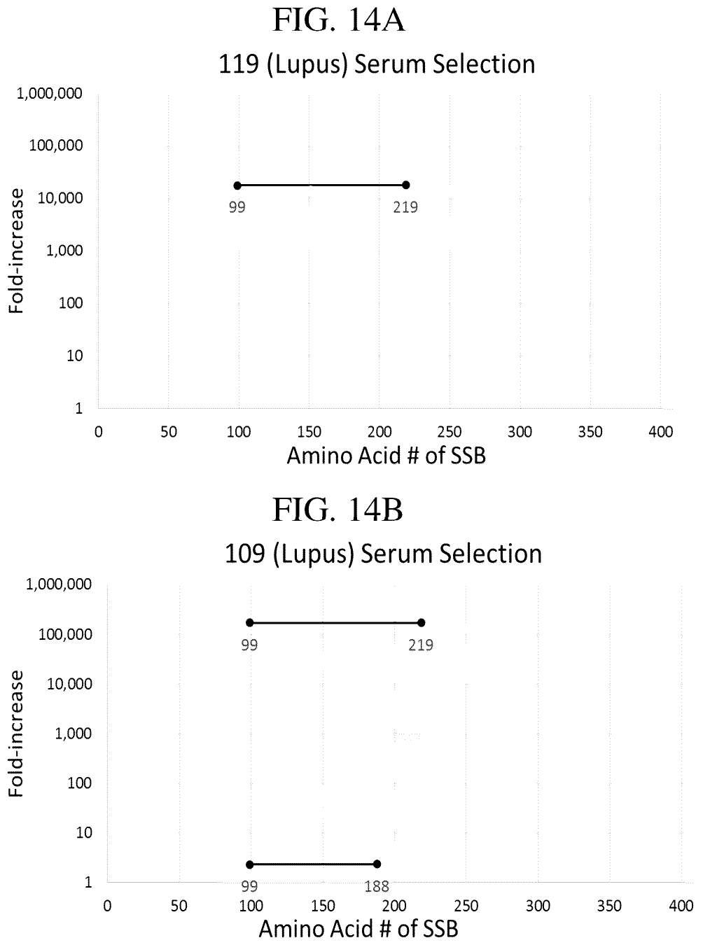

FIG. 14 illustrates the preferential selection of dominant SSB domains after immunoselection of antigen display libraries with antibodies from serum samples 119 and 109. SSB/La-specific autoantibodies in serum from SLE patient 119 predominantly reacted with a protein fragment (fragment 99-219) containing the RRM1 domain (amino acids 111-187) while SSB/La-specific autoantibodies in serum from SLE patient 109 predominantly reacted with two protein fragments encoding the RRM1 domain (fragment 99-219 and 99-188). Serum 119 selection resulted in a >10,000-fold increase in fragment counts relative to the fragment counts present within the unselected antigen display libraries. Most domain counts decrease substantially in frequency due to extensive washing during the selection assays. Serum 109 selection resulted in an 120,000-fold increase in fragment counts relative to the fragment counts present within the unselected antigen display libraries. Thereby, it is likely that reactivity of the ELISA negative serum samples from patients 109 and 112 with domain fragment 99-219 of SSB is due to the exposure of protein epitopes that are normally concealed by domains flanking either side of the RM1 domain in the full-length SSB protein. Alternatively, adherence of the full-length SSB/La protein to plastic in the ELISA format may conceal or denature the autoantibody-binding epitope(s) identified by autoantibodies in these two sera. Either way, the identification and utilization of protein domains that are identified by autoantibodies may have advantages over the use of intact or immobilized full-length proteins in some diagnostic assays. Moreover, the current antigen display and selection system allows the generation of libraries with unparalleled diversity of conformational epitopes for antibody identification and quantification. As demonstrated in this example with the SSB autoantigen, the current antigen display and selection system also has the benefit of simultaneous domain and epitope mapping, which may have additional diagnostic and discovery benefits.

1. An antigen display library comprising a Ff phage-based library comprised of a plurality of phage clones containing DNA inserts inserted therein, wherein the DNA inserts:
(a) are derived from mRNA from a cell type or tissue type;
(b) comprise an average length selected from between about 150 nucleotides and about 900 nucleotides;
(c) are selected for in-frame expression as part of a gene; and
wherein the diversity of antigenic epitopes encoded by the DNA inserted in the phage library comprising the antigen display library is estimated to be greater than 1×10⁶.

2. An antigen display library comprising a plurality of clones containing a plurality of DNA inserts inserted therein, wherein the DNA inserts:
(a) each encode a polypeptide;
(b) comprise an average length selected from between about 150 nucleotides and about 900 nucleotides;
(c) are selected for in-frame expression of the polypeptide; wherein the clones are optionally expressed in a phage-based library, and wherein the diversity of polypeptides encoded by the DNA inserts in the antigen display library is greater than 1×10⁶.

3.-21. (canceled)


SEQUENCE LISTING (18 sequences)

SEQ ID NO: 1 - DNA, 20 bp, synthetic construct
  misc_feature 1..20: note = Synthetic primer
  misc_feature 12..20: note = n is a, c, g, or t
  source 1..20: mol_type = other DNA
gcggccgcaacnnnnnnnnn

SEQ ID NO: 2 - DNA, 23 bp, synthetic construct
  misc_feature 1..23: note = Synthetic primer
  misc_feature 15..23: note = n is a, c, g, or t
  source 1..23: mol_type = other DNA
tggccgccgagaacnnnnnnnnn

SEQ ID NO: 3 - DNA, 46 bp, synthetic construct
  misc_feature 1..46: note = Synthetic primer
  source 1..46: mol_type = other DNA
gctggtggtgccgttctatagccatagcaccatggccgccgagaac

SEQ ID NO: 4 - DNA, 45 bp, synthetic construct
  misc_feature 1..45: note = Synthetic primer
  source 1..45: mol_type = other DNA
ttttactttcaccagcgtttctgggtgagctgcagcggccgcaac

SEQ ID NO: 5 - DNA, 54 bp, synthetic construct
  misc_feature 1..54: note = Synthetic primer
  source 1..54: mol_type = other DNA
taaacaactttcaacagtttcagctctgatatctttggatccagcggccgcaac

SEQ ID NO: 6 - DNA, 50 bp, synthetic construct
  misc_feature 1..50: note = Synthetic primer
  source 1..50: mol_type = other DNA
ccgctggcttgctgctgctggcagctcagccggccatggccgccgagaac

SEQ ID NO: 7 - DNA, 46 bp, synthetic construct
  misc_feature 1..46: note = Synthetic primer
  source 1..46: mol_type = other DNA
gttccagtgggtccggatacggcaccggcgcaccggcggccgcaac

SEQ ID NO: 8 - DNA, 52 bp, synthetic construct
  misc_feature 1..52: note = Synthetic primer
  source 1..52: mol_type = other DNA
tggcgtaacacctgctgcaaatgctgcgcaacacgccatggccgccgagaac

SEQ ID NO: 9 - DNA, 18 bp, synthetic construct
  misc_feature 1..18: note = Synthetic primer
  source 1..18: mol_type = other DNA
cggccgctggatccaaag

SEQ ID NO: 10 - DNA, 18 bp, synthetic construct
  misc_feature 1..18: note = Synthetic primer
  source 1..18: mol_type = other DNA
ccatggccggctgagctg

SEQ ID NO: 11 - DNA, 23 bp, synthetic construct
  misc_feature 1..23: note = Synthetic primer
  source 1..23: mol_type = other DNA
gcggccgccggtgcgccggtgcc

SEQ ID NO: 12 - DNA, 25 bp, synthetic construct
  misc_feature 1..25: note = Synthetic primer
  source 1..25: mol_type = other DNA
ccatggcgtgttgcgcagcatttgc

SEQ ID NO: 13 - DNA, 80 bp, synthetic construct
  misc_feature 1..80: note = Synthetic primer
  misc_feature 25..30: note = n is a, c, g, or t
  source 1..80: mol_type = other DNA
caagcagaagacggcatacgagatnnnnnngtgactggagttcagacgtgtgctcttccgatcaatccagcggccgcaac

SEQ ID NO: 14 - DNA, 75 bp, synthetic construct
  misc_feature 1..75: note = Synthetic primer
  source 1..75: mol_type = other DNA
aatgatacggcgaccaccgagatctacactctttccctacacgacgctcttccgatctccatggccgccgagaac

SEQ ID NO: 15 - DNA, 77 bp, synthetic construct
  misc_feature 1..77: note = Synthetic primer
  misc_feature 25..30: note = n is a, c, g, or t
  source 1..77: mol_type = other DNA
caagcagaagacggcatacgagatnnnnnngtgactggagttcagacgtgtgctcttccgatcccggcggccgcaac

SEQ ID NO: 16 - DNA, 24 bp, synthetic construct
  misc_feature 1..24: note = Synthetic primer
  source 1..24: mol_type = other DNA
ccgatctccatggccgccgagaac

SEQ ID NO: 17 - DNA, 24 bp, synthetic construct
  misc_feature 1..24: note = Synthetic primer
  source 1..24: mol_type = other DNA
tccgatcaatccagcggccgcaac

SEQ ID NO: 18 - DNA, 20 bp, synthetic construct
  misc_feature 1..20: note = Synthetic primer
  source 1..20: mol_type = other DNA
ccgatcccggcggccgcaac